(19) United States
(12) Patent Application Publication
(10) Pub. No.: US 2018/0207078 A1
(43) Pub. Date: Jul. 26, 2018

(54) COMBINATION OF NATURAL SUBSTANCES COMPRISING AT LEAST ONE GLYCYRRHETINIC ACID AND AT LEAST ONE GUGGULSTERONE, AND USE THEREOF FOR COSMETIC APPLICATIONS

(71) Applicant: OntoChem, GmbH, Halle/Saale (DE)

(72) Inventors: Lutz Weber, Germering (DE); Robert Rennert, Halle/Saale (DE)

(21) Appl. No.: 15/336,764

(22) PCT Filed: Apr. 13, 2016
(86) PCT No.: PCT/EP2016/057995

§ 371 Date: Oct. 27, 2016

(30) Foreign Application Priority Data
Apr. 13, 2015 (EP) EP 15 001 045.2
Nov. 5, 2015 (EP) EP 15 003 170.6

(51) Int. Cl. A61K 8/63; A61K 8/55; A61K 8/37; A61K 8/34; A61K 8/9789; A61Q 19/06; A61Q 19/08; A61K 8/02

(52) U.S. Cl. CPC A61K 8/63; A61K 8/553; A61K 8/37; A61K 8/34; A61K 2800/5922; A61K 8/9789; A61Q 19/06; A61Q 19/08; A61K 8/0212; A61K 8/375

(57) ABSTRACT

The present invention relates to a combination of natural substances comprising at least one glycyrrhetinic acid and at least one guggulsterone, and to the use thereof for cosmetic applications. The present invention relates especially to vesicles (especially for topical application) comprising at least one glycyrrhetinic acid and at least one guggulsterone. The vesicles are preferably added to a cream, a lotion or a gel, the resulting cream, lotion or gel being used for the cosmetic and/or dermatological treatment of the skin and/or for the prophylaxis of the appearance of cellulite and/or ageing skin and/or for the treatment of changes in subcutaneous fatty or connective tissue, for example lipomas and other non-specific subcutaneous fat deposits, or for the regression or reduction of fat pads which are not caused by disease, such as, for example, fat pads in the face and neck region, for example lachrymal sacs, etc.

This application is a continuation of International Application No. PCT/EP2016/057995 (WO2016/166091) with an International Filing Date of Apr. 12, 2016, which claims under 35 U.S.C. § 119 the benefit of European Application No. 15 001 045.2, filed Apr. 13, 2015 and European Application No. 15 003 170.6, filed Nov. 5, 2015, the entire contents of which are incorporated herein by reference.

The present invention relates to vesicles (especially for topical application) comprising at least one glycyrrhetinic acid and at least one guggulsterone. The vesicles are preferably added to a cream, a lotion or a gel, the resulting cream, lotion or gel being used for the cosmetic and/or dermatological treatment of the skin and/or for the prophylaxis of the appearance of cellulite and/or ageing skin. The cream, lotion or gel can comprise further auxiliary substances (excipients) which, for example, improve the effectiveness, the storage life, the odour or other properties of the cream, lotion or gel as a skin care product. The vesicular formulation of the active ingredient combination comprising the at least one glycyrrhetinic acid and the at least one guggulsterone ensures sufficient topical penetration and thus the desired cosmetic and/or dermatological effect on cellulite and/or ageing skin.

The present invention relates further to a face mask (for example an anti-ageing face mask) comprising at least one glycyrrhetinic acid and at least one guggulsterone. The face mask according to the invention preferably comprises vesicles according to the invention having at least one glycyrrhetinic acid and at least one guggulsterone.

The present invention relates further to a plaster or patch (for example for the treatment of double chin, lipomas, etc.) comprising at least one glycyrrhetinic acid and at least one guggulsterone. The plaster or patch according to the invention preferably comprises vesicles according to the invention having at least one glycyrrhetinic acid and at least one guggulsterone.

The effective cosmetic and/or dermatological treatment and/or prophylaxis of the appearance of cellulite or of ageing skin continues to be a problem which has not been solved satisfactorily. Cellulite in particular is a cosmetic and perception problem in about 80% of all women, resulting mainly from the characteristic structure of female connective tissue in conjunction with subcutaneous fat stores. As a result of a weakening of the connective tissue, the causes of which can be, for example, genetic, age-related, the result of an increase in weight or pregnancy, the subcutaneous fat deposits, especially where there are large fat stores, are visible on the skin surface as characteristically uneven “orange peel”. One possible method of treating cellulite is thus to shape the cutaneous and subcutaneous fat layers, that is to say reduce excessive fat stores by inhibiting adipocyte differentiation, breaking down existing fat deposits by increasing lipolysis in the adipocytes and inducing apoptosis in adipocytes, as well as promoting fatty tissue homoeostasis.

Numerous compositions have been described as preparations for this purpose, especially topical formulations in a cream, a lotion or a gel. In most cases, the active principle is combinations especially of natural substances or plant extracts, such as, for example:
the combination of ivy, gingko and horsetail extract
green coffee, cocoa, seaweed (Fucus vesiculosus and Odontella aurita), also as a drink
the combination of lotus extract and carnitine (Beiersdorf, Hamburg)
cocoa polyphenols, yacon leaf extract (Nuxe, Paris)
Brazilian mimosa with extract of yellow horned-poppy (Nuxe, Paris).

In many cases, the treatment, in accordance with the supplier's instructions, must be supported by, for example, wrapping, massage or even riding. Clinical observations are described in only a very small number of cases, for example for a combination of 10 essential oils (strawflower, cedar, grapefruit, etc.) from Nuxe (Paris). In a clinical study which was carried out under dermatological supervision in 31 women after 56 days of twice-daily application, a reduced abdominal belt was observed in the participants and the hip measurement was reduced on average by up to 1.5 cm in 33% of the participants (Nuxe, Paris). However, since cellulite affects the upper thighs in particular, it would have been interesting to measure the change in the circumference of the upper thigh. In a double-blind study of the circumference of the upper thigh in 18 women (9 treated/9 placebo), topically applied 2.5% glycyrrhetinic acid in a cream (on the market as Minus Adip® cream) was used on 9 women; although the thickness of the superficial fat layer on the upper thighs was reduced slightly, the circumferences of the upper thighs could scarcely be reduced (Armanini D. et al., Steroids 2005, 70, 538-542).

In general, all the compositions hitherto available and tested have been described as inadequate in tests relating to their anti-cellulite action (Stiftung Warentest, Issue 5, 2009), which in most cases might be attributable to a low biochemical activity of the natural substances used (at least at a dose which can be used without risk), coupled with insufficient penetration of the active ingredients through the skin barrier—especially through the stratum corneum.

Although other compositions are promoted with dermatologically tested activity, they are not safe because of the expected side-effects. Quadrollol® (Q-L®) from Erlacos (Erlangen), for example, consists of a so-called bi-complex of phytosterols (cholesterol derivatives) and ER1446 (17β-O-acetyl-4-hydroxy-androsten-3-one), an esterified testosterone derivative, which as well as having anabolic properties also has androgenic properties, which can be effective in the treatment of cellulite in women but, because of the expected androgenic side-effects, appear undesirable.

WO 2010/121814 (L. Weber and T. Wilckens) describes the use of an 11βHSD1 inhibitor, such as, for example, 18β-glycyrrhetinic acid, and an inhibitor of the mineral corticoid receptor, such as, for example, Z-guggulsterone, as a pharmaceutical combination for the treatment of various diseases, for example tumours, but also cellulite. However, in that patent application, the pharmaceutical formulation of the claimed combination is not implemented with any example, either as a tablet, injection, suppository or as a topical formulation. Likewise, no usable doses are indicated. It is known from toxicological investigations that 18β-glycyrrhetinic acid in the form of glycyrrhizine can safely be administered orally or by another systemic route to an average human being only up to a daily dose of approximately 16 mg (R. A. Isbrucker, G. A. Burdock, Regulatory Toxicology and Pharmacology 2006, 46, 167-192). It has been found, however, that this systemic maximum dose is far too low to accumulate in the skin the concentration necessary for the effective treatment of cellulite and/or ageing skin. Furthermore, a systemic administration of more than 30 mg/day of guggulsterone is not safe, because hepatitis has been observed in one case with Equisterol, a preparation comprising guggulsterone (Grieco A. et al., Journal of Hepatology 2009, 50(6), 1273-1277).

Accordingly, the object of the present invention is to provide tolerable formulations (especially for topical application, such as, for example, a cream, a lotion or a gel) which, especially by shaping of the fat layers of the skin, permit cosmetic and/or dermatological treatment and/or prophylaxis of the appearance of ageing skin or cellulite more effectively than hitherto.

In order to transport the substances used for the treatment of cellulite to their active sites in deeper skin layers, it is first necessary in the case of topical application to penetrate the upper skin layers (stratum corneum, epidermis, dermis) efficiently, which for most natural substances is a problem that is not easy to solve owing to the natural barrier function especially of the stratum corneum. Because of the special nature of the upper skin layers that are to be penetrated, the active ingredients are typically not transported into deeper skin layers by conventional and known cosmetic and topical formulations, for example in a typical cream (Minus Adip®, Newfields, Paris), but have a penetration depth of only up to 10 μm (Elias, P. M., Friends, D. S., J. Cell Biol. 1975, 20, 1-19).

Numerous methods are known to the expert for penetrating the stratum corneum for a possible cellulite treatment, for example the use of ionophoresis, electrochemical methods, radiowaves, ultrasound or microneedles. However, these methods are not easy to carry out in daily use, especially if they are not carried out in a health care clinic, and are therefore not suitable here especially where daily application is necessary. Another possibility is the use of penetration enhancers such as DMSO, propylene glycol or urea. Both glycyrrhetinic acid and guggulsterone can in fact penetrate into deeper skin layers with DMSO, but regular/daily use of DMSO as a penetration enhancer is not advisable owing to the known side-effects of DMSO (see negative decision of the United States Environmental Protection Agency on DMSO, 16 Jun. 2006). Unlike DMSO, urea and propylene glycol alone do not exhibit sufficient activity as a penetration enhancer for glycyrrhetinic acid or guggulsterone.

Other methods for improving skin penetration recommend formulating the active ingredients in a lipid encapsulation which is based, for example, for the so-called liposomes on lecithin or the main constituent thereof, phosphatidylcholine (Artmann C. et al., Arzneimittelforschung 1990, 40 (12): 1365-1368; Blume G., Skin Delivery Systems 2008, ed. J. W. Wiechers, Allured Publishing Corp. USA, 269-282). It has been found that liposomes produced using egg lecithin have a size of approximately 200 nm, and glycyrrhetinic acid encapsulated in such liposomes is not transported into deeper skin layers. However, an anti-inflammatory effect was achieved in the upper skin layers.

Also suitable in principle for dermal active ingredient transport are the so-called transfersomes, a combination of lipid components with surface-active substances, for example Tween 80, which yield highly flexible vesicles which, when formulated and applied as a cream, for example, are able to transport the enclosed active ingredients into deep skin layers (Lampen P. et al., J. Cosmet. Sci. 2003, 54: 119-131; Zhang Y.-T. et al., Int. J. Pharm. 2014, 471: 449-452; Li C. et al., Int. J. Nanomed. 2013, 8: 1285-1292; Badran M. et al., Scientific World Journal 2012, Art. ID: 134876; Cevc G. et al., Adv. Drug Deliv. Rev. 1996, 18: 349-378). It has likewise been shown that even very large molecules, for example the protein insulin, were transported by means of transfersomes transdermally through the skin into the systemic blood stream and consequently caused a significant systemic effect, in the case of insulin a reduction in the blood glucose level (Cevc G. et al., Biochim. Biophys. Acta 1998, 1368: 201-215). On the basis of this finding, the use of transfersomes as transport vesicles for the combination of glycyrrhetinic acid and guggulsterone is not preferred because the target site of transfersomes can scarcely be limited to the skin layers to be treated and any systemic administration of the active ingredient combination that is to be applied dermally is undesirable, in order to rule out any systemic side-effects.

A further modification of conventional liposomes are the so-called ethosomes, which in principle are also suitable for dermal active ingredient transport. Ethosomes generally comprise 1-5% phosphatidylcholine/lecithin and 20-50% ethanol. Their good skin-penetrating action is based in particular on the penetration-enhancing action of the high ethanol content (Romero E. L. et al., Int. J. Nanomed. 2013, 8: 3171-3181; Touitou E. et al., J. Contr. Rel. 2000, 65: 403-418). However, the skin tolerance of ethosomes is very controversial. The high ethanol content can increase the solubilisation of the protective lipid layer of the stratum corneum and thus the transdermal moisture loss of the skin (Blume G., Skin Delivery Systems 2008, ed. J. W. Wiechers, Allured Publishing Corp. USA, 269-282). Ethosomes are thus not suitable for daily use and especially extensive use on the upper thigh. In addition, a high ethanol content cannot easily be formulated as a cream, a lotion or a gel with pleasant sensory properties (for example odour). The use of ethosomes as transport vesicles for the combination of glycyrrhetinic acid and guggulsterone is therefore not preferred.

It has now been found, surprisingly, that vesicles based on a lipidic emulsifier from the food additives group, a monoester of a long-chained fatty acid (for example an oleate) and optionally a plant oil are suitable for the encapsulation of the active ingredient combination of at least one glycyrrhetinic acid and at least one guggulsterone. This formulation allows lipophilic but also hydrophilic substances to be encapsulated even with higher packaging rates than in lecithin-containing liposomes.

Also surprisingly, vesicles having an active ingredient combination of at least one glycyrrhetinic acid and at least one guggulsterone are suitable for transport into deeper skin layers if they are based on phosphatidylcholine (preferably phosphatidylcholine from soy lecithin or sunflower lecithin). As compared with egg lecithin, soy and sunflower lecithin, or especially soy and sunflower phosphatidylcholine, are distinguished by a particularly high content of unsaturated fatty acids, for example diunsaturated linoleic acid. It has further been found, surprisingly, that phosphatidylcholines that contain unsaturated fatty acids and have a phase transition below 35° C. are especially suitable for transporting the active ingredient combination into the deeper skin layers. Such fatty acids are especially linoleic acid, alpha-linolenic acid, palmitoleic acid or oleic acid.

Furthermore, it has been found that especially vesicles having a phosphatidylcholine content of ≥80% by weight transport the active ingredient combination surprisingly efficiently through the stratum corneum as well as through the upper skin layers into the subcutaneous fatty tissue.

The combination of a glycyrrhetinic acid with a guggulsterone in the vesicles according to the invention surprisingly exhibits a significantly improved activity profile, especially in respect of the treatment and/or prophylaxis of cellulite, as compared with the use of glycyrrhetinic acid or guggulsterone alone. Both natural substances surprisingly exhibit a synergistically enhanced effect in combination.

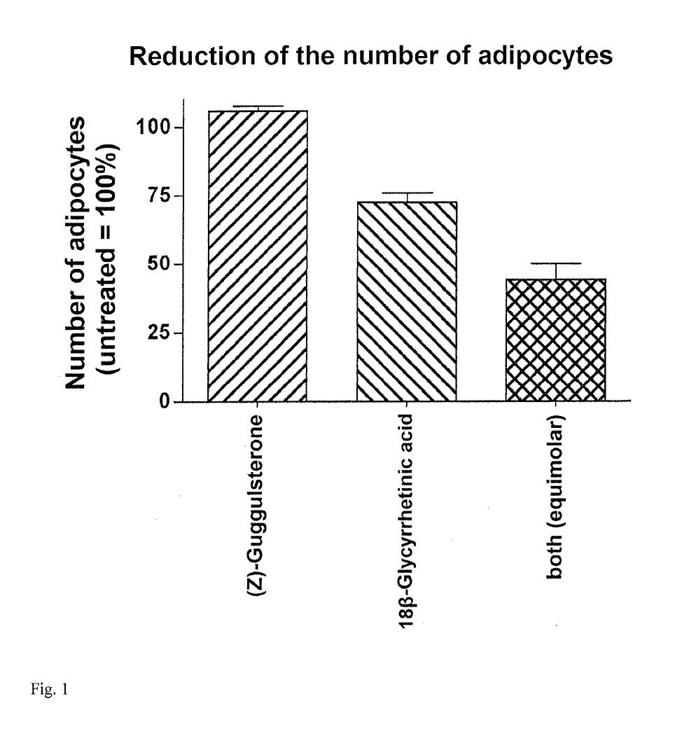

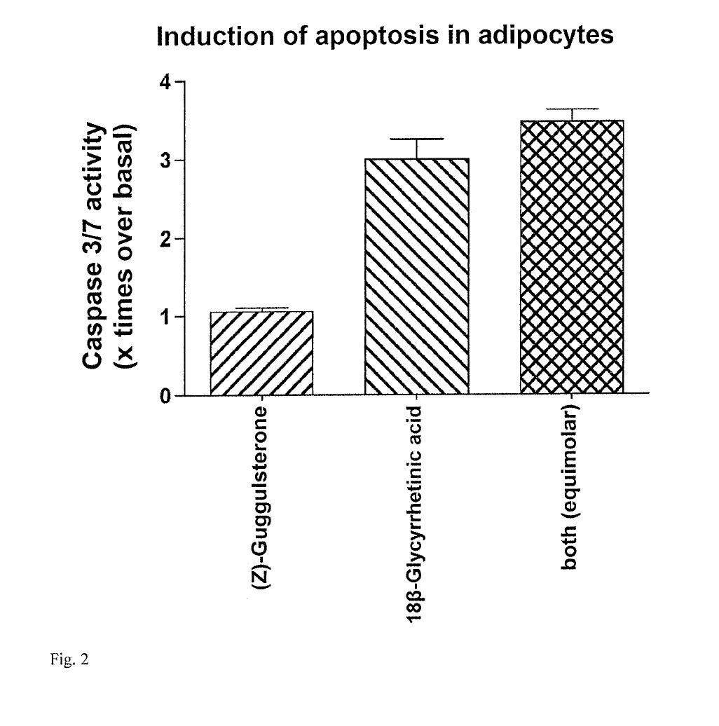

Guggulsterone inhibits lipid storage in maturing adipocytes significantly better than does glycyrrhetinic acid (see Table 1). Surprisingly, guggulsterone in combination with glycyrrhetinic acid also synergistically enhances the positive effects of glycyrrhetinic acid on the induction of adipocyte apoptosis and thus on the reduction of the number of adipocytes in the fatty tissue (see FIG. 1 and FIG. 2). Furthermore, guggulsterone in combination with glycyrrhetinic acid surprisingly enhances the lipolytic action of glycyrrhetinic acid on mature adipocytes. This means that a combination of a glycyrrhetinic acid derivative and a guggulsterone derivative surprisingly reduces both the fat content of maturing and mature adipocytes and the number of adipocytes itself significantly more efficiently than either of the two natural substances could on its own.

However, the use and effectiveness of both glycyrrhetinic acid and guggulsterone in water-based formulations for topical application, such as creams, lotions or gels, is limited according to the current prior art by the poor solubility of both natural substances in water.

A further disadvantage in the previous application especially of glycyrrhetinic acid according to the current prior art is the inadequate penetration of the existing glycyrrhetinic-acid-containing skin care products, which is due, for example, to their poor water solubility in combination with the special barrier function of the skin.

By encapsulating glycyrrhetinic acid and guggulsterone in the vesicles according to the invention it is readily possible, surprisingly, to incorporate them in demonstrably effective concentrations in aqueous formulations for topical application, whereby the critical disadvantages of poor water solubility and inadequate topical penetration according to the current prior art are remedied.

The present invention provides inter alia a topical formulation, such as, for example, a cream, a lotion or a gel, which comprises the vesicles according to the invention.

The present invention relates to vesicles (especially for topical application) comprising:

a) at least one glycyrrhetinic acid or a salt or an ester thereof;

b) at least one guggulsterone; and

c) either: c1) phosphatidylcholine (preferably phosphatidylcholine from soy lecithin or sunflower lecithin), or
c2) at least one emulsifier from the food additives group and at least one monoester of a long-chained fatty acid.

The present invention relates especially to vesicles comprising:

a) at least one glycyrrhetinic acid or a salt or an ester thereof;

b) at least one guggulsterone; and

c) phosphatidylcholine (preferably phosphatidylcholine from soy lecithin or sunflower lecithin).

The present invention further relates especially to vesicles comprising:

a) at least one glycyrrhetinic acid or a salt or an ester thereof;

b) at least one guggulsterone; and

c) at least one emulsifier from the food additives group and at least one monoester of a long-chained fatty acid.

The present invention further relates especially to vesicles comprising:

a) at least one glycyrrhetinic acid or a salt thereof;

b) at least one guggulsterone; and

c) phosphatidylcholine (preferably phosphatidylcholine from soy lecithin or sunflower lecithin).

The present invention further relates especially to vesicles comprising:

a) at least one glycyrrhetinic acid or a salt thereof;

b) at least one guggulsterone; and

c) at least one emulsifier from the food additives group and at least one monoester of a long-chained fatty acid.

The present invention further relates preferably to vesicles which further comprise a plant oil (vegetable oil), wherein the plant oil is preferably a seed oil of an edible plant, such as, for example, sunflower oil (Helianthus annuus), rape oil (Brassica napus) or soybean oil (Glycine max).

The at least one glycyrrhetinic acid or the salt or the ester thereof is preferably selected from: 18α-glycyrrhetinic acid and/or 18β-glycyrrhetinic acid or one or more salt(s) or ester(s) thereof.

Further preferably, the at least one guggulsterone is selected from (Z)-guggulsterone and/or (E)-guggulsterone.

Further preferably, the at least one glycyrrhetinic acid or the salt or the ester thereof is used in the form of Glycyrrhiza glabra or in the form of extract from Glycyrrhiza glabra and/or in the form of a pure chemical substance and/or in the form of salts or esters thereof.

Further preferably, the at least one guggulsterone is used in the form of guggul lipid or in the form of extract from guggul lipid and/or in the form of a pure chemical substance.

The vesicles of the present invention are preferably unilamellar.

Further preferably, the phosphatidylcholine of the vesicles according to the invention originates from soy lecithin or sunflower lecithin.

Furthermore, the phosphatidylcholine preferably contains two unsaturated fatty acids, for example linoleic acid, oleic acid or alpha-linolenic acid.

Furthermore, the vesicles preferably comprise at least 80% by weight phosphatidylcholine.

Particularly preferably, the vesicles according to the invention have a particle size of 20-200 nm, preferably of 50-100 nm.

Furthermore, the vesicles of the present invention particularly preferably have a polydispersity index of less than 0.25.

Before the vesicles according to the invention are formulated as a cream, lotion or gel, they are preferably extruded through a PC filter membrane (preferably having a pore size of not more than 200 nm, particularly preferably not more than 100 nm pore size) or are treated by an alternative standard method, known to the person skilled in the art, for the corresponding size limitation of lipid vesicles, for example dialysis using a membrane or a filter (preferably having a pore size of not more than 200 nm, particularly preferably not more than 100 nm pore size) or gel chromatography (for example with Sephadex G75).

Alternatively, the vesicles according to the invention can also comprise only a glycyrrhetinic acid (or a salt or an ester thereof) or a guggulsterone. In that case, however, there are then added to the final formulation both the vesicles that comprise a glycyrrhetinic acid (or a salt or an ester thereof) and the vesicles that comprise a guggulsterone, in the form of a mixture in the ratio of preferably from 2:1 to 1:2 (especially 1:1).

According to one embodiment, the present invention relates to a composition comprising:

A) vesicles comprising:

a) at least one glycyrrhetinic acid or a salt or an ester thereof; and

c) either: c1) phosphatidylcholine, or
c2) at least one emulsifier from the food additives group and at least one monoester of a long-chained fatty acid; and

B) vesicles comprising:

b) at least one guggulsterone; and

d) either: d1) phosphatidylcholine, or
d2) at least one emulsifier from the food additives group and at least one monoester of a long-chained fatty acid.

Particularly preferably, the present invention relates to vesicles comprising:
0.1-0.5 part by weight of a glycyrrhetinic acid or of a salt or an ester thereof (especially 18β-glycyrrhetinic acid);
0.1-0.5 part by weight of a guggulsterone (especially (Z)-guggulsterone); and
0.5-3 parts by weight of at least one surfactant (especially cholesterol, sodium cholate and/or sodium deoxycholate);

per 10 parts by weight of phosphatidylcholine, preferably from soy or sunflower lecithin.

Further particularly preferably, the present invention relates to vesicles comprising:

15-20% (v/v) seed oil of an edible plant (for example sunflower seed oil (Helianthus annuus));

2.5-10% (w/v) emulsifiers from the food additives group (for example Imwitor® 375 (glyceryl citrate/lactate/linoleate/oleate; Cremer Oleo GmbH, Hamburg));

2.5-5% (w/v) of at least one monoester of a long-chained fatty acid (for example polyglyceryl-2 oleate);

10-20% (v/v) ethanol;

0.1-10% (w/v) 18β-glycyrrhetinic acid; and

0.1-10% (w/v) (Z)-guggulsterone;

made up to 100% with water (preferably aqua bidest.).

Further particularly preferably, the present invention relates to vesicles comprising:

7-17.5% (w/v) emulsifiers from the food additives group (for example Imwitor® 375 (glyceryl citrate/lactate/linoleate/oleate; Cremer Oleo GmbH, Hamburg));

3-7.5% (w/v) of at least one monoester of a long-chained fatty acid (for example ethyl oleate);

10-20% (v/v) ethanol;

0.1-10% (w/v) 18β-glycyrrhetinic acid; and

0.1-10% (w/v) (Z)-guggulsterone;

made up to 100% with water (preferably aqua bidest.).

According to a preferred embodiment, the present invention relates to vesicles based on a lipidic emulsifier from the food additives group, a monoester of a long-chained fatty acid and a plant oil and comprising 18β-glycyrrhetinic acid and (Z)-guggulsterone. Alternatively, the vesicles can also comprise only 18β-glycyrrhetinic acid or (Z)-guggulsterone. In that case, however, there are then added to the final formulation both the vesicles that comprise 18β-glycyrrhetinic acid and the vesicles that comprise (Z)-guggulsterone, in the form of a mixture in the ratio of preferably from 2:1 to 1:2 (especially 1:1).

Further particularly preferably, the present invention relates to vesicles comprising:

0-5% (w/w) glycyrrhetinic acid or a salt or an ester thereof

0-5% (w/w) guggulsterone or a salt or an ester thereof

5-25% (w/w) abs. ethanol

1-30% (w/w) ethyl oleate

0-30% (w/w) plant oil (for example sunflower oil, soybean oil, rape oil)

0.5-5% (w/w) diglyceryl monooleate

0-5% (w/w) Imwitor 375 (glyceryl citrate/lactate/linoleate/oleate)

ad 100% purified water;

wherein the vesicles comprise at least glycyrrhetinic acid or a salt or an ester thereof or guggulsterone or a salt or an ester thereof.

According to a further preferred embodiment, the present invention relates to vesicles based on a lipidic emulsifier from the food additives group and a monoester of a long-chained fatty acid and comprising an active ingredient combination of 18β-glycyrrhetinic acid and (Z)-guggulsterone. Alternatively, the vesicles can also comprise only 18β-glycyrrhetinic acid or (Z)-guggulsterone. In that case, however, there are then added to the final formulation both the vesicles that comprise 18β-glycyrrhetinic acid and the vesicles that comprise (Z)-guggulsterone, in the form of a mixture in the ratio of preferably from 2:1 to 1:2 (especially 1:1).

Further particularly preferably, the present invention relates to vesicles comprising:

0-5% (w/w) glycyrrhetinic acid or a salt or an ester thereof

0-5% (w/w) guggulsterone or a salt or an ester thereof

5-25% (w/w) abs. ethanol

1-10% (w/w) ethyl oleate

5-25% (w/w) Imwitor 375 (glyceryl citrate/lactate/linoleate/oleate)

ad 100% purified water;

wherein the vesicles comprise at least glycyrrhetinic acid or a salt or an ester thereof or guggulsterone or a salt or an ester thereof.

According to a further preferred embodiment, the present invention relates to vesicles based on synthetic cetyl palmitate, a plant oil (for example jojoba oil, sunflower seed oil, soybean oil, rape oil), a plant polysaccharide copolymer as surface-active matrix stabiliser or a derivative thereof (for example inulin, or Inutec® SP1, microcrystalline cellulose, or Avicel PH105, etc.) and a stearic-acid-based emulsifier (for example TEGO® Care PS, TEGO® Care PSC-3, TEGO® Care 450, etc.) and comprising an active ingredient combination of 18β-glycyrrhetinic acid and (Z)-guggulsterone. Alternatively, the vesicles can also comprise only 18β-glycyrrhetinic acid or (Z)-guggulsterone. In that case, however, there are then added to the final formulation both the vesicles that comprise 18β-glycyrrhetinic acid and the vesicles that comprise (Z)-guggulsterone, in the form of a mixture in the ratio of preferably from 2:1 to 1:2 (especially 1:1).

Further particularly preferably, the present invention relates to vesicles comprising:

0-5% (w/w) glycyrrhetinic acid or a salt or an ester thereof

0-5% (w/w) guggulsterone or a salt or an ester thereof

10-15% (w/w) jojoba oil

15-25% (w/w) cetyl palmitate

0.2-1.0% (w/w) Inutec® SP1

1-3% (w/w) TEGO® Care PS

ad 100% purified water;

wherein the vesicles comprise at least glycyrrhetinic acid or a salt or an ester thereof or guggulsterone or a salt or an ester thereof.

The vesicles of the present invention are preferably formulated as a cream, a lotion or a gel.

Further preferably, the cream, the lotion or the gel of the present invention is characterised in that it comprises an active ingredient combination of at least one glycyrrhetinic acid or a salt or an ester thereof and at least one guggulsterone each in an amount of 0.01-10% (w/w), preferably 0.1-5% (w/w), based on the total weight of the cream, lotion or gel.

The present invention relates further to the use of the vesicles according to the invention in the production of a cream, a lotion or a gel for the treatment and/or prophylaxis of cellulite.

The present invention relates further to the use of the vesicles according to the invention in the production of a cream, a lotion or a gel for the treatment and/or prophylaxis of the appearance of ageing skin.

The use of the vesicles according to the invention comprising at least one glycyrrhetinic acid and at least one guggulsterone in a cream, lotion or gel permits surprisingly efficient vesicular transport of the active ingredient combination into deeper, subcutaneous skin layers, whereby a direct and quantifiable action on the maturing and mature adipocytes of the subcutaneous fat deposits is, surprisingly, achieved. Furthermore, it has been found, surprisingly, that it is possible by means of the present invention to reduce the number of adipocytes in the cutaneous and subcutaneous fatty tissue in a lasting manner—as a result of inhibited adipogenesis and the induction of apoptosis in mature adipocytes—which brings about an improvement in the appearance and elasticity of the skin and is thus of particular interest for the treatment and/or prophylaxis of cellulite (tightening of “orange peel”). The use of such a cream, lotion or gel according to the invention additionally improves the moisture balance and reduces the roughness of the skin.

The vesicles according to the invention are preferably lipidic transport vesicles.

Within the scope of the present invention, the term vesicle denotes especially spherical or roundish to oval arrangements of lipids (especially amphiphilic lipids) and/or emulsifiers in an aqueous medium. Vesicles according to the invention can especially absorb lipophilic, hydrophilic and/or amphiphilic compounds in their membrane shell or in their lumen. The membrane shell can consist of a lipid double layer or a lipid monolayer and can enclose an aqueous lumen or a lumen consisting of a plant oil or of both.

Within the scope of the present invention, the emulsifiers from the food additives group are especially esters or ester mixtures of one or more polyhydroxy compound(s) (such as glycerol, a pentose, a hexose or a polysaccharide) with in each case one or more unsaturated fatty acid(s) (such as oleic acid, linoleic acid or alpha-linolenic acid) and optionally one or more short-chained (C₂₋₆) organic acid(s) (especially a mono-, di- or tri-acid having from 2 to 6 carbon atoms and optionally substituted by one or more OH group(s) or acetyl group(s), such as, for example, acetic acid, lactic acid, citric acid, tartaric acid or diacetyltartaric acid). Examples are mono- or di-glycerides or sugar fatty acid esters or corresponding mixtures of the two, such as sugar glycerides. Examples of sugar glycerides are sucrose glycerides, as well as mixtures with above-mentioned esters. Special preference is given to esters and ester mixtures comprising diacylglycerides.

Preferred emulsifiers from the food additives group are the food additives E471 to E477 (E471, E472a, E472b, E472c, E472d, E472e, E472f, E473, E474, E475, E476 and E477) as are disclosed, for example, in EU regulation no. 1130/2011 of 11 Nov. 2011 or in the Liste des aid infodienstes e.V. (as at March 2014).

According to the invention, a long-chained fatty acid is a preferably unbranched fatty acid having from 12 to 24 carbon atoms and from 0 to 6 double bonds. The monoester of a long-chained fatty acid is preferably an ester of a long-chained fatty acid with a mono- or polyhydroxy compound, such as, for example, ethanol, glycerol, a pentose, a hexose or a polysaccharide.

Examples of monoesters of a long-chained fatty acid are sucrose oleate, trehalose isostearate, glyceryl monooleate, diglyceryl oleate, polyglyceryl-2 oleate, polyglyceryl-4 oleate, propylene glycol oleate, ethyl oleate or the corresponding esters of linoleic acid, alpha-linolenic acid, eicosapentaenoic acid or stearic acid. Special preference is given to the ethyl and polyglyceryl esters of the unsaturated long-chained fatty acids, such as oleic acid, linoleic acid, alpha-linolenic acid or eicosapentaenoic acid. The monoester of a long-chained fatty acid preferably has a membrane-stabilising action.

Within the scope of the present invention, the term lecithin denotes especially a mixture, obtained from a natural plant source, of phosphoglycerides such as phosphatidylcholines, phosphatidylethanolamines, phosphatidylserines, phosphatidylinositols, sphingocholines and glycolipids, wherein both the percentage composition of the phosphoglycerides and the nature and percentage composition of the esterified fatty acids can vary in dependence on the plant source.

Within the scope of the present invention, phosphatidylcholine denotes specifically only the phosphoglycerides consisting of glycerol, fatty acids, phosphoric acid and choline, which can be obtained from natural plant sources, preferably soybean or sunflower oil or lecithin, or can also be prepared synthetically. While phosphatidylcholine from plant sources is generally a mixture of phosphatidylcholines with different fatty acid esterifications, the nature and percentage composition of the esterified fatty acids varying in dependence on the plant source, synthetic phosphatidylcholines can contain a defined combination of two fatty acids, preferably two unsaturated fatty acids such as oleic acid, linoleic acid or alpha-linolenic acid.

According to the invention, synthetic cetyl palmitate denotes a mixture of esters of natural saturated and mono- or di- or tri-unsaturated C12-C24 fatty acids with saturated C12-C24 fatty acid alcohols having a mean molecular weight of ˜500 g/mol, comprising, for example, the eponymous esters of saturated palmitic acid (C16) and saturated cetyl alcohol (C16).

According to the invention, plant polysaccharide copolymers as surface-active matrix stabilisers are polysaccharide derivatives, for example fructan derivatives or microcrystalline cellulose derivatives. Examples of fructan derivatives are inulin—a mixture of fructose polymers of up to 100 fructose molecules and a terminal glucose molecule—and derivatives of inulin, such as, for example, Inutec® SP1. Examples of microcrystalline cellulose, that is to say partially depolymerised α-cellulose, are the Avicel PH celluloses.

According to the invention, stearic-acid-based emulsifiers are mono- or di-esters of stearic acid with polyglycerol—preferably polyglyceryl-3—and/or methylglucose. Examples of such stearic-acid-based emulsifiers are methylglucose sesquistearate (TEGO® Care PS), polyglyceryl-3 dicitrate stearate (TEGO® Care PSC-3) and polyglyceryl-3 methylglucose distearate (TEGO® Care 450).

Guggul (synonyms: loban, bdellium resin, bedellium, bdellium, gum guggulu, Indian myrrh) is the resin of the balsam tree Commiphora mukul.

A principal constituent of the guggul lipid are the guggulsterones, which occur in the (E) and (Z) configuration. It has not hitherto been possible to observe a significant difference in biological activity between the two forms.

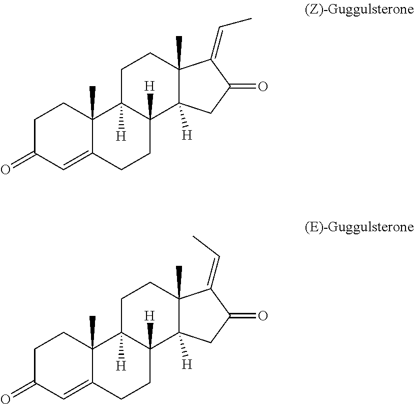

Liquorice (Glycyrrhiza glabra) is a plant from the subfamily of the papilionaceous plants (Faboideae). Liquorice contains glycyrrhizine, a mixture of potassium and calcium salts of glycyrrhizinic acid. By cleaving the diglucuronide of glycyrrhizinic acid there are formed from glycyrrhizine the glycyrrhetinic acids (GA)—a mixture of 18β-glycyrrhetinic acid and, in a smaller proportion, 18α-glycyrrhetinic acid.

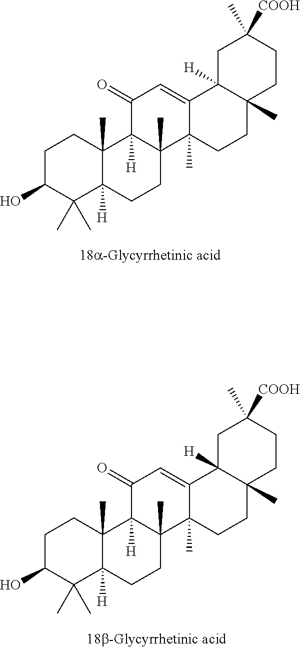

Preferred salts of the glycyrrhetinic acids are alkali or alkaline earth salts, such as, for example, sodium, potassium, lithium, calcium or magnesium salts, ammonium salts or salts of organic bases, such as, for example, methylamine, dimethylamine, triethylamine, piperidine, ethylenediamine, lysine, choline hydroxide, meglumin, morpholine or arginine.

Preferred esters of the glycyrrhetinic acids are esters of saturated, straight-chained or branched alcohols having from 1 to 18 carbon atoms. Particularly preferred esters are methyl esters, glyceryl esters and stearyl esters.

The vesicles according to the invention preferably comprise surfactants, which can be used especially for modifying the properties of the vesicles, such as, for example, cholesterol, sodium cholate or sodium deoxycholate.

According to a preferred embodiment of the invention, a composition according to the invention, for example in the form of a cream, a lotion or a gel, can comprise excipients such as carrageen, 1,2-hexanediol, butylene glycol dicaprylate, butylmethoxydibenzoylmethane, shea butter, acrylate/C10-C30 alkyl acrylate crosspolymer, C12-C15 alkyl benzoate, caprylic acid triglyceride, capryloylglycerol/sebacic acid, TEGO® Carbomer 140, TEGO® Care PS, Carbopol 981, carboxymethylcellulose, cetearyl alcohol, cetyl palmitate, cetyl alcohol, cetyl stearyl alcohol, citric acid, decyl oleate, dibutyl adipate, diethylaminohydroxybenzoylhexyl benzoate, diheptyl oleate, dimethicone, sodium EDTA and disodium EDTA, Flowerconcentrole®, ethylhexylglycerol, ethylhexyl laurate, ethylhexyl salicylate, ethylhexyl stearate, ethyl octanoate, glycerol, glyceryl monostearate, glyceryl stearate citrate, urea, hydrogenated coco-glycerides, Inutec® SP1, isopropyl palmitate, isooctyl laurate, jojoba oil, potassium sorbate, Keltrol CG-SFT, limonene, linanool, Macrogol 20 glyceryl monostearate, magnesium sulfate heptahydrate, mannitol, methyl hydroxybenzoate, methylparaben, methylpropanediol, Miglyol 812, sodium chloride, sodium edetate, sodium hyaluronate, sodium hydroxide, octyldecanol, panthenol, viscous paraffin, perfume, pentylene glycol (Hydrolite), Pionier 1033, polyoxyethylene laurate ester (PEG-6 L), phenoxyethanol, phenylbenzimidazolesulfonic acid, Phospholipon, propylene glycol, propyl hydroxybenzoate, polyacrylic acid, polyvinylpyrrolidone, sodium stearyl glutamate, SLM 2026, stearyl alcohol, squalane, sucrose stearate, triglycerol diisostearate, trisodium ethylenediamine, beeswax, sodium carbomer, ethanol, unguentum emulsificans aquosum, vasilinum album (white petroleum jelly), vitamin E acetate, water (aqua bidest.), xanthan gum or other suitable excipients known to the expert.

The vesicles according to the invention can be processed by known methods to a cream, a lotion or a gel.

Particular preference is given according to the invention to relatively small, unilamellar vesicles with high fluidity and flexibility of shape. These vesicles particularly preferably have a particle size of 20-200 nm (especially 50-100 nm), a polydispersity index <0.25 (both detected by measuring the dynamic light scattering (DLS), for example using a Zetasizer Nano ZS, Malvern Instruments, Germany) and a high inclusion rate for both natural substances, since such vesicles surprisingly exhibit a very advantageous ability to permeate human skin while at the same time having negligible toxicity and irritation to the skin, as well as surprisingly high particle stability in a formulation as a cream, lotion or gel.

According to the invention, the above-described requirements of particle size, stability, skin penetration and side-effects profile (for example skin irritation, risk of systemic side-effects) are met especially by flexible vesicles containing soy or sunflower lecithin or soy or sunflower phosphatidylcholine (especially an amount of at least 80% by weight phosphatidylcholine and a large amount of unsaturated fatty acids) or flexible vesicles based on a lipidic emulsifier from the food additives group, a monoester of a long-chained fatty acid, for example an oleate, and optionally a plant oil.

A further aspect of the present invention relates to a face mask (for example an anti-ageing face mask) which comprises at least one glycyrrhetinic acid or a salt or an ester thereof and at least one guggulsterone. The face mask according to the invention preferably comprises a vesicle according to the invention having at least one glycyrrhetinic acid or a salt or an ester thereof and at least one guggulsterone.

The face mask of the present invention is preferably characterised in that it comprises an active ingredient combination of at least one glycyrrhetinic acid or a salt or an ester thereof and at least one guggulsterone each in an amount of 0.01-5% (w/w), preferably 0.1-2.5% (w/w), based on the total weight of the face mask.

The present invention relates further to the use of the vesicles according to the invention in the production of a face mask for the treatment and/or prophylaxis of ageing or stressed skin.

Further preferred changes to the subcutaneous fatty or connective tissue which can be treated according to the invention are, for example, lipoedema, lipoma, lipomatosis of the abdominal wall, dermatopanniculosis deformans, pseudogynecomastia, buffalo hump in HIV patients, and non-specific subcutaneous fat deposits. Furthermore, fat pads which are not caused by disease, such as, for example, fat pads in the face and neck region (for example lachrymal sacs, nasolabial folds, flabby cheeks, double chin, etc.), can be made to regress or can be reduced according to the invention. The present invention relates further to the use of the vesicles according to the invention or of a combination of at least one glycyrrhetinic acid (or a salt or an ester thereof) and at least one guggulsterone for after-treatment following liposuction.

A further aspect of the present invention relates to a plaster or patch which comprises at least one glycyrrhetinic acid or a salt or an ester thereof and at least one guggulsterone. The plaster or patch according to the invention preferably comprises a vesicle according to the invention having at least one glycyrrhetinic acid and at least one guggulsterone.

Alternatively, a plaster or patch according to the invention can comprise vesicles according to the invention which each comprise only a glycyrrhetinic acid (or a salt or an ester thereof) or a guggulsterone. In that case, however, there are then added to the plaster or patch both the vesicles that comprise a glycyrrhetinic acid (or a salt or an ester thereof) and the vesicles that comprise a guggulsterone, in the form of a mixture in the ratio of preferably from 2:1 to 1:2 (especially 1:1).

Such a plaster or patch can comprise, for example, (i) a top layer or covering layer, (ii) optionally a removable protective layer and (iii) one or more active-ingredient-containing layers or reservoirs each arranged between the top layer and the optional removable protective layer.

The expression “active-ingredient-containing layer or reservoir” denotes a layer or a reservoir which comprises at least one glycyrrhetinic acid or a salt or an ester thereof and/or at least one guggulsterone. The active-ingredient-containing layer or reservoir preferably comprises a vesicle according to the invention having at least one glycyrrhetinic acid and at least one guggulsterone, or a mixture of vesicles according to the invention each comprising only a glycyrrhetinic acid (or a salt or an ester thereof) or a guggulsterone. The active-ingredient-containing layer or reservoir preferably comprises a formulation of a cream, a gel or a lotion according to the invention.

The plaster or patch can further have on the skin side an adhesive layer or an adhesive layer carried by the covering layer. In the plaster or patch according to the invention, the adhesive layer can be a self-adhesive polymer matrix layer.

In a particular embodiment of the invention, the adhesive layer has an opening or is in non-adhesive form in at least one region. The adhesive layer preferably does not extend over the active-ingredient-containing layer.

The plaster or patch according to the invention can comprise from 0.1 to 1000 mg, especially from 1 to 500 mg, preferably from 1 to 250 mg, of each of glycyrrhetinic acid and guggulsterone.

The plaster or patch according to the invention having the one or more active-ingredient-containing layer(s) or the one or more reservoirs can be, for example, a matrix system or a reservoir system.

In the plaster or patch according to the invention, the at least one glycyrrhetinic acid and/or the at least one guggulsterone can be present in one or more active-ingredient-containing layers or in one or more active-ingredient-containing reservoirs; preferably in the form of a formulation as a cream, gel or lotion according to the invention.

The adhesive layer can consist of or comprise any skin-compatible adhesive which is approved for dermal use. Such adhesive systems, for example based on polymers forming a gel in water, polyisobutylene, cataplasms, polyacrylic acid or polyacrylates, are known to the person skilled in the art. The adhesive layer is preferably water-resistant, in order to prevent the plaster from slipping under the effect of perspiration.

The adhesive layer in the plaster or patch according to the invention can further comprise at least one component or consist of a component which is selected from the group of natural rubber, synthetic rubber, polyacrylate, polyvinyl acetate, polyisobutylene, silicone, especially polydimethylsiloxane, and hydrogel, especially high molecular weight polyvinylpyrrolidone, polyvinyl alcohol and oligomeric polyethylene oxide or mixtures thereof.

The adhesive layer in the plaster or patch according to the invention can especially comprise or consist of a polyacrylate.

In the plaster or patch according to the invention, the polyacrylate can comprise one or more monomer units which are derived from one or more monomers selected from the group of n-butyl acrylate, isobutyl acrylate, propyl acrylate, methyl acrylate, 2-ethylhexyl acrylate, 2-hydroxyethyl acrylate and 2-hydroxyethyl methacrylate.

The synthetic rubber in the plaster or patch according to the invention can be a styrene-butadiene-styrene block copolymer or a styrene-butadiene block copolymer.

The active-ingredient-containing layer(s) and/or the adhesive layer in the plaster or patch according to the invention can further comprise a crosslinker.

The active-ingredient-containing layer in the plaster or patch according to the invention can further be a matrix of a material which can be made to start to dissolve or can be dissolved by skin moisture.

The plaster or patch according to the invention can further comprise the at least one glycyrrhetinic acid and/or the at least one guggulsterone in a hydrogel. Furthermore, the plaster or patch according to the invention can comprise the at least one glycyrrhetinic acid and/or the at least one guggulsterone in the form of a formulation as a cream, gel or lotion according to the invention.

The plaster or patch according to the invention can be characterised by hydroxymethylcellulose, hydroxypropylcellulose, carbopol or polyvinyl alcohol as the hydrogel.

The plaster or patch according to the invention can further comprise a thickener or swelling agent, preferably hydroxypropylcellulose, especially Klucel.

The plaster or patch according to the invention can further comprise one or more permeation promoters. The plaster or patch according to the invention can thus comprise at least one permeation promoter from the group of ethyl alcohol, isopropyl alcohol, octylphenol, polyethylene glycol (PEG), especially PEG400, pentylene glycol, propylene glycol, polyethylene glycol octylphenyl ether, oleic acid, linoleic acid, linolenic acid, jojoba oil, soybean oil, aloe vera extract, dodecanol, urea, Labrafil, Labrasol, fatty acid esters, especially isopropyl myristate, monolaurates, preferably methyl laurate, propylene glycol monolaurate, glycerol monolaurate and polyethylene glycol monolaurate, octyldodecyl lactate, glycerol monooleate and glycol monooleate, Transcutol, tocopherol, tocopheryl acetate, panthenol, N-decylmethylsulfoxide, triacetin and N-methylpyrrolidone.

The plaster or patch according to the invention can further comprise one or more preservatives, especially from the group of alcohols, quaternary amines, organic acids, parabens and phenols.

The plaster or patch according to the invention can further comprise one or more supersaturation stabilisers, for example polyvinylpyrrolidone.

The plaster or patch according to the invention can further be characterised by a covering film consisting of or comprising a material from the group of polyolefin, polyester, polyvinylidene chloride, polyurethane, cotton, viscose or wool. The covering film preferably consists of or comprises polyester.

The top layer can further consist of or comprise, for example, coated paper, polysilicone, polyethylene, polypropylene, copolymers of ethylene and vinyl acetate, polyesters, polyurethanes, polyamide, polyacrylate and polyisobutylene. The top layer can be used in the form of a closed or perforated film, a woven fabric or a closed or perforated nonwoven.

The plaster or patch according to the invention can be characterised by a covering film having a thickness of from 0.01 to 1.5 mm and especially from 0.03 to 1.0 mm.

Plasters according to the invention can have a size of from 1 to 50 cm² and can be rectangular, square, round, elliptical or trapezoidal. The plasters are preferably square with an edge length of approximately 1-5 cm or rectangular with an edge length of 1-10 cm.

The plaster or patch according to the invention can further have a surface area of from 2 to 100 cm², preferably from 5 to 50 cm², especially from 10 to 40 cm², for example approximately 20 cm².

Finally, the plaster or patch according to the invention can be provided with an overtape.

The top layer, the adhesive layer and/or the matrix layer can comprise aromatic oils or perfumes. Those layers can further be permeable to air and moisture in order to improve the wear comfort.

According to a preferred embodiment, the at least one active-ingredient-containing layer comprises a vesicle or a vesicle mixture according to the invention.

According to a further preferred embodiment, the present invention relates to micelles which comprise at least one glycyrrhetinic acid or a salt or an ester thereof and at least one guggulsterone. Alternatively, the micelles according to the invention can also comprise only a glycyrrhetinic acid (or a salt or an ester thereof) or a guggulsterone. In that case, however, there are then added to the final formulation both the micelles that comprise a glycyrrhetinic acid (or a salt or an ester thereof) and the micelles that comprise a guggulsterone, in the form of a mixture in the ratio of preferably from 2:1 to 1:2 (especially 1:1). Examples of such micelles are polymer micelles of a block copolymer having a hydrophilic segment and a hydrophobic segment.

The “block copolymer having a hydrophilic segment and a hydrophobic segment” described in the present invention preferably means a copolymer which can be present in an aqueous medium preferably in the form of a polymer micelle of the core (which comprises mainly hydrophobic segments)-shell (which comprises mainly hydrophilic segments) type. The “hydrophilic segment” which forms such a block copolymer includes poly(ethylene oxide), poly(malic acid), poly(saccharide), poly(acrylic acid), poly(vinyl alcohol) and poly(vinylpyrrolidone) segments. The “hydrophobic segment” includes segments of polyamino acids. The “hydrophobic segment” includes especially poly-(β-benzyl aspartate), poly-(γ-benzyl glutaminate), poly-(β-alkyl aspartate), poly(lactide), poly-(ε-caprolactone), poly-(δ-valerolactone), poly-(γ-butyrolactone) and poly-(α-amino acid) segments as well as two or more types thereof.

According to the invention, the micelles of the present invention, like the vesicles according to the invention or together therewith, can be formulated as a cream, a lotion, a gel or a face mask or can be used in a plaster or patch according to the invention.

### EXAMPLES

Cell Culture

The 3T3-L1 mouse fibroblasts were cultivated in Dulbecco's modified Eagle's medium (DMEM) incl. 10% foetal calf serum (FCS) and 2 mM L-glutamine at 37° C., 95% humidity and 5% CO₂.

Adipocyte differentiation and maturation was induced as described (Yang J. Y. et al., Obesity 2008, 16, 16-22; Shugart E. C. and Umek R. M., Cell Growth & Differentiation 1997, 8, 1091-1098). The cells were stimulated not only with dexamethasone, as is generally conventional, but also with cortisol, which is more physiological.

The cells were seeded into 96-well plates and cultivated to confluence. Two days after reaching cell confluence (D0=day 0), cell differentiation was induced by adding DMEM with 10% FCS, 167 nM insulin, 0.5 μM IBMX and 1 μM dexamethasone or 200 nM cortisol for two days (D2). The cells were then cultivated for a further two days in DMEM with 10% FCS and 167 nM insulin (D4), followed by cultivation in DMEM/10% FCS for a further four days (D8). Ultimately, >20-30% of the cells were mature adipocytes with accumulated lipid stores.

Determination of the Cellular Lipid Content

As an indicator for the degree of adipogenesis, the lipid content of the cells (intracellular lipid droplets) was detected by means of the lipophilic colour reagent Nile red, as described (Yang J. Y. et al., Obesity 2008, 16, 16-22; Greenspan P., The Journal of Cell Biology 1985, 100, 965-973). 3T3-L1 cells (5,000-10,000 cells/well), after reaching confluence, were cultivated for a further two days in 96-well plates (D0). The cells were then treated with guggulsterone and/or glycyrrhetinic acid, individually or in combination, for days D0-D2, D2-D4, D4-D6 and D0-D6 of the adipogenesis (concentration range: 1-200 μM). The medium, with or without test substance(s), was changed or renewed every two days. Cells treated as follows served as controls: (1) pure cell culture medium (undifferentiated control), (2) pure differentiation medium (untreated control), and (3) DMSO, corresponding to the highest DMSO concentration in the test batches, in differentiation medium (DMSO control).

On day 6 (D6), the intracellular lipid accumulation (lipid droplets) was quantified by means of a Nile red assay. To that end, the cell medium was discarded and the cells were washed with 100 μl/well of PBS (pH 7.4). The cells were then incubated for 20 minutes at room temperature with 50 μl/well of Nile red in PBS (10 μg/ml). The lipid content was determined fluorometrically using a Synergy 2 plate reader (BioTek, Winooski, USA) (excitation: 485 nm, emission: 560 nm, mirror: 550 nm). Nile red selectively stains neutral lipids, for example triacylglycerides in intracellular droplets.

For analyses by fluorescence microscopy, the cells were treated on Lab-Tek™ chamber slides (Nunc, Langenselbold, Germany) as described above and incubated for days D2-D4 of the adipogenesis with guggulsterone and/or glycyrrhetinic acid (50 or 100 μM; individually or in equimolar combination). The incubation medium was then discarded and the cells were washed with PBS and stained with Nile red (10 μg/ml) and DAPI (1 μg/ml) in 50 μl/well of PBS for 20 minutes at room temperature. After removal of the staining solution, the cells were washed with PBS and fixed with 4% paraformaldehyde in PBS for 30 minutes at room temperature. Finally, the cells were washed three times with PBS and mounted on the object slide by means of Fluorescence Mounting Medium (Dako, Carpinteria, USA). The fluorescence microscopy images were taken by using a Zeiss Axio Observer.Z1 with ApoTome (Carl Zeiss Microlmaging, Jena, Germany) using the DAPI and GFP filter settings.

Cell Viability Assay

The cell viability of mature adipocytes after treatment with a test compound was measured by means of a fluorimetric Resazurin-based assay. Vital, that is to say metabolically active, cells are capable of converting Resazurin into the fluorescent derivative Resorufin (Strotmann U. J. et al., Ecotox. Environ. Safety 1993, 25, 79-89).

3T3-L1 cells were seeded into 96-well plates (5,000-10,000 cells/well) and, as described above, cultivated to maturity (D8). On D8, the mature adipocytes were incubated for 24 hours under standard culture conditions with guggulsterone and/or glycyrrhetinic acid (1-250 μM). Cells treated as follows served as controls: (1) pure cell culture medium (undifferentiated control), (2) pure differentiation medium (untreated control), (3) DMSO, corresponding to the highest DMSO concentration in the test batches, in differentiation medium (DMSO control), and (4) 150 μg/ml digitonin in DMEM (cell death control). After 24 hours, the incubation medium was discarded and the cells were washed with 100 μl/well of DMEM and then incubated for 2 hours under standard culture conditions with 50 μl/well of Resazurin solution (60 μM in DMEM). Finally, the Resorufin fluorescence was detected by means of a Synergy 2 plate reader (BioTek, Winooski, USA) (excitation: 540 nm, emission: 590 nm, mirror: 550 nm).

Apoptosis Assay

In order to evaluate a possible induction of apoptosis in mature adipocytes as a result of the treatment with (Z)-guggulsterone and/or glycyrrhetinic acid, a Caspase-Glo® 3/7 assay kit (Promega, Mannheim, Germany) was used.

3T3-L1 cells were seeded into 96-well plates (5,000-10,000 cells/well) and, as described above, cultivated to maturity (D8). On D8, the mature adipocytes were incubated for 24 hours under standard culture conditions with guggulsterone and/or glycyrrhetinic acid (1-250 μM). Cells treated as follows served as controls: (1) pure cell culture medium (undifferentiated control), (2) pure differentiation medium (untreated control), and (3) DMSO, corresponding to the highest DMSO concentration in the test batches, in differentiation medium (DMSO control). After 24 hours, the incubation medium was discarded, the cells were washed with 100 μl/well of DMEM, and then 150 μl/well of Caspase-Glo® 3/7 reagent in DMEM (1:1, v/v) were added. After incubation for 2 hours at room temperature, the luminescence signal of the reagent was detected by means of a Synergy 2 plate reader (BioTek, Winooski, USA).

Lipolysis Assay

Lipolysis of mature adipocytes as a result of the treatment with guggulsterone and/or glycyrrhetinic acid was detected by means of a glycerol release assay. For this purpose, the glycerol released into the cell medium as a result of the lipolysis of the cells was quantified using a free glycerol staining reagent (Cayman Chemical, Ann Arbor, USA).

3T3-L1 cells were seeded into 96-well plates (5,000-10,000 cells/well) and, as described above, cultivated to maturity (D8). On D8, the mature adipocytes were incubated for 24 hours under standard culture conditions with guggulsterone and/or glycyrrhetinic acid (1-250 μM). Cells treated as follows served as controls: (1) pure cell culture medium (undifferentiated control), (2) pure differentiation medium (untreated control), and (3) DMSO, corresponding to the highest DMSO concentration in the test batches, in differentiation medium (DMSO control).

Following the incubation, 25 μl of the cell medium of each sample were transferred into a corresponding well of a new 96-well plate and incubated with 90 μl of the free glycerol staining reagent for 15 minutes at room temperature. 25 μl samples with 90 μl of aqua dest. instead of the reagent served as the measurement reference. Finally, the content of released glycerol was determined on the basis of the absorption at 540 nm by means of a Synergy 2 plate reader (BioTek, Winooski, USA).

Lipid Content in Pre-Adipocytes

The lipid content, that is to say the storage of triacylglycerides in intracellular lipid droplets, is an indicator for the degree of adipogenesis. We used a Nile red assay to study the effects of (Z)-guggulsterone and 18β-glycyrrhetinic acid, respectively, on the dexamethasone- or cortisol-stimulated adipogenesis of 3T3-L1 mouse fibroblasts. Since the staining reagent Nile red, upon excitation at 485 nm and fluorescence detection at 560 nm, is very selective for neutral lipids, it was thus possible specifically to measure the intracellular accumulation of triacylglycerides during advancing adipogenesis.

The period of treatment within the 6-day differentiation period of the adipocytes was very important for the effects of (Z)-guggulsterone or 18β-glycyrrhetinic acid on cellular lipid accumulation which are to be observed. Both compounds, (Z)-guggulsterone and 18β-glycyrrhetinic acid, inhibited the lipid accumulation of the cells to the greatest extent when they were applied over the entire 6-day differentiation period. Microscopic analyses illustrated very clearly the effects of (Z)-guggulsterone and 18β-glycyrrhetinic acid on the lipid content of 3T3 pre-adipocytes which were treated with 100 μM (Z)-guggulsterone and 100 μM 18β-glycyrrhetinic acid, respectively, over the period D0-D6 of the adipogenesis (stimulation with 1 μM dexamethasone). Stimulated but untreated cells served as reference and showed a high degree of lipid storage. By contrast, after treatment of the cells with (Z)-guggulsterone and 18β-glycyrrhetinic acid, respectively, significantly fewer and smaller lipid droplets were to be detected.

For the most part, this effect was induced in days D0-D2, that is to say the initial phase of the adipocyte differentiation. The reduction of the lipid content of correspondingly treated 3T3 pre-adipocytes, 0-100 μM (Z)-guggulsterone and 18β-glycyrrhetinic acid, respectively, in the period D0-D2, is summarised in the following table:

TABLE 1

| | (Z)-Guggulsterone | | | | 18β-Glycyrrhetinic acid | | | |
|---|---|---|---|---|---|---|---|---|
| | 0 | 10 | 40 | 100 | 0 | 10 | 40 | 100 |
| | μM | μM | μM | μM | μM | μM | μM | μM |
| Lipid | 100% | 71% | 26% | 5% | 100% | 98% | 91% | 7% |
| content* | | | | | | | | |

*normalised against unstimulated/untreated cells

On day 6 of the adipogenesis (D6), the accumulation of intracellular lipid droplets was measured fluorometrically by means of a Nile red assay (excitation: 485 nm; emission: 560 nm; 550 nm mirror). The data were normalised and the lipid accumulation is given in percent; unstimulated cells were used as 0% reference, and stimulated but untreated cells were used as 100% reference.

On treatment of the cells for the entire 6-day adipocyte differentiation (D0-D6), an IC₅₀ value for the inhibition of cellular lipid accumulation of ˜5 μM was obtained for (Z)-guggulsterone and of ˜20 μM for 18β-glycyrrhetinic acid. Both compounds, (Z)-guggulsterone and 18β-glycyrrhetinic acid, thus inhibit the accumulation of triacylglycerides in 3T3-L1 cells during adipogenesis, but guggulsterone is slightly more potent than glycyrrhetinic acid in this respect. In the activity range of 18β-glycyrrhetinic acid (≥50 μM), a synergistic action was additionally detected in conjunction with (Z)-guggulsterone used in an equimolar amount.

Treatment of the cells in the middle differentiation phase (days D2-D4) likewise brought about good inhibition of lipid accumulation. For stimulation of adipocyte differentiation, cortisol, which is more physiological, was also used instead of the conventional dexamethasone for comparison, no substantial differences being observed as regards the results.

Cell Viability/Number of Mature Adipocytes

Fluorometric Resazurin-based cell viability assays were carried out in order to investigate possible effects of a treatment with (Z)-guggulsterone and/or 18β-glycyrrhetinic acid on the viability, and thus the number, of mature 3T3 adipocytes. To that end, mature 3T3 adipocytes were stimulated with 1 μM dexamethasone and then treated for 24 hours under standard culture conditions with increasing concentrations of (Z)-guggulsterone or 18β-glycyrrhetinic acid or of an equimolar mixture of both compounds. Finally, the cell viability, as a measure of the number and fitness of the cells, was measured by means of a Resazurin assay.

As is shown in FIG. 1, a 24-hour treatment with up to 250 μM (Z)-guggulsterone did not impair the cell viability. 250 μM 18β-glycyrrhetinic acid, on the other hand, reduced the cell viability, that is to say the number of mature adipocytes, by 28% with the same treatment time. Treatment of the cells with equimolar mixtures of both compounds, (Z)-guggulsterone and 18β-glycyrrhetinic acid, resulted in a significant effect synergy. The combination of 250 μM of each of the two compounds reduced the cell viability by 56%. (Z)-Guggulsterone—although without effect on its own—appears to be capable of enhancing the cytostatic effect caused by 18β-glycyrrhetinic acid on mature adipocytes.

Induction of Apoptosis in Mature Adipocytes

Possible reasons for the observed decrease in cell viability may be anti-proliferative effects of the test compound (that is to say induction of a cell cycle arrest), a reduction in the metabolic activity of the cells, or a reduction of the number of vital adipocytes following the induction of apoptosis.

In order to test whether the effects of 18β-glycyrrhetinic acid, either alone or in equimolar combination with (Z)-guggulsterone, in reducing cell viability may be based on the induction of apoptosis, correspondingly treated adipocytes were investigated in respect of an activation of the effector caspases 3/7 (in the case of advanced apoptosis). Regardless of the type of adipocyte stimulation, with dexamethasone or with cortisol, (Z)-guggulsterone showed no induction of caspases 3/7. By contrast, treatment of the cells with 250 μM of 18β-glycyrrhetinic acid for 24 hours brought about an increase in caspase 3/7 activity by a factor of 2.5-3.0 as compared with untreated cells. The equimolar combination of both compounds, (Z)-guggulsterone and 18β-glycyrrhetinic acid, showed—in line with the results of the viability assay—a synergistic effect of the two compounds in respect of caspase activation. The combination of 250 μM of each of the two compounds brought about an increase in caspase 3/7 activity by a factor of 3.5-4.0 as compared with untreated cells (FIG. 2).

In summary, 18β-glycyrrhetinic acid inhibits the viability of mature adipocytes by inducing an apoptotic cell death programme as a result of which, inter alia, caspases 3/7 are activated. Although (Z)-guggulsterone on its own is inactive in this context, it is capable of enhancing the effects of 18β-glycyrrhetinic acid.

Lipolysis in Mature Adipocytes

In order to study a possible degradation of the intracellular triacylglyceride reservoirs in mature adipocytes, that is to say lipolysis, as a result of treatment with (Z)-guggulsterone and/or 18β-glycyrrhetinic acid, a glycerol release assay was carried out. The enzymatic degradation of triacylglycerides leads to the production of glycerol, which is membrane-permeable and is thus released into the surrounding culture medium. Free glycerol in the medium can be quantified spectrophotometrically by means of specific staining reagents. After 24 hours' treatment with the equimolar combination (in each case 250 μM) of the two compounds, (Z)-guggulsterone and 18β-glycyrrhetinic acid, the degradation of intracellular triglycerides had increased by ˜66% as compared with an untreated control.

The examples which follow are intended to illustrate exemplary embodiments of the present invention.

### Example 1: Flexible Soy-Phosphatidylcholine-Containing Liposomes, Comprising the Active Ingredient Combination of 18β-Glycyrrhetinic Acid and (Z)-Guggulsterone

For the preparation of flexible soy-phosphatidylcholine-containing liposomes, soy phosphatidylcholine (Phospholipon 90 G; Lipoid, Ludwigshafen) and surfactants, such as, for example, cholesterol or sodium cholate, as well as 18β-glycyrrhetinic acid and (Z)-guggulsterone were mixed in the weight ratio 10/0.5-3/0.1-0.5/0.1-0.5 (w/w/w/w). The preferred formulation comprises Phospholipon® 90 G (≥95% soy phosphatidylcholine), sodium cholate, 18β-glycyrrhetinic acid and (Z)-guggulsterone in the ratio 10/1/0.3/0.3 (w/w/w/w). Lipid and surfactant were dissolved in chloroform/MeOH (1:2 v/v) and concentrated to dryness in vacuo at 40° C. overnight in a rotary evaporator. The resulting lipid film was rehydrated (1 h/30° C./200 rpm) with PBS (pH 7.4), which contained the corresponding amounts of glycyrrhetinic acid and guggulsterone, to give a 10% lipid suspension. The suspension was treated with ultrasound for 20 minutes (on/off interval: 10 s) and extruded through a PC filter membrane (100 nm pore size). The vesicle size was analysed by means of dynamic light scattering using a Zetasizer Nano ZS (Malvern Instruments GmbH, Herrenberg, Germany; for 5 minutes at room temperature) and was in the range 50-100 nm.

### Example 2: Vesicles Based on a Lipidic Emulsifier from the Food Additives Group, a Monoester of a Long-Chained Fatty Acid and a Plant Oil and Comprising an Active Ingredient Combination of 18β-Glycyrrhetinic Acid and (Z)-Guggulsterone

For the preparation of oil emulsifier vesicles containing glycyrrhetinic acid and guggulsterone, 15-20% (v/v) of sunflower seed oil (Helianthus annuus); 2.5-10% (w/v) Imwitor® 375 (glyceryl citrate/lactate/linoleate/oleate; Cremer Oleo GmbH, Hamburg); 2.5-5% (w/v) polyglyceryl-2 oleate; 10-20% (v/v) ethanol were mixed with 0.1-10% (w/v) of each of 18β-glycyrrhetinic acid and (Z)-guggulsterone and made up to 100% with aqua bidest. The preferred formulation comprises 20% (v/v) sunflower seed oil; 5% (w/v) Imwitor® 375; 2.5% (w/v) polyglyceryl-2 oleate; 15% (v/v) ethanol; 5% (w/v) 18β-glycyrrhetinic acid and 5% (w/v) (Z)-guggulsterone in aqua bidest. The sunflower seed oil, Imwitor® 375, polyglyceryl-2 oleate, 18β-glycyrrhetinic acid and (Z)-guggulsterone were dissolved at room temperature in ethanol, with shaking (500 rpm). Under constant conditions, aqua bidest. was then added slowly and evenly (200 μl/min) to 100% (v/v), and the whole was mixed thoroughly. Finally, the dispersion was vortexed for 5 minutes, treated with ultrasound for 5 minutes (on/off interval: 10 s) and extruded through a PC filter membrane (100 nm pore size). The vesicle size was analysed by means of dynamic light scattering using a Zetasizer Nano ZS (Malvern Instruments GmbH, Herrenberg, Germany; for 5 minutes at room temperature) and was in the range 80-100 nm.

### Example 3: Vesicles Based on a Lipidic Emulsifier from the Food Additives Group and a Monoester of a Long-Chained Fatty Acid and Comprising an Active Ingredient Combination of 18β-Glycyrrhetinic Acid and (Z)-Guggulsterone

For the preparation of emulsifier vesicles containing glycyrrhetinic acid and guggulsterone, 7-17.5% (w/v) Imwitor® 375 (glyceryl citrate/lactate/linoleate/oleate; Cremer Oleo GmbH, Hamburg); 3-7.5% (w/v) ethyl oleate; 10-20% (v/v) ethanol were mixed with 0.1-10% (w/v) of each of 18β-glycyrrhetinic acid and (Z)-guggulsterone and made up to 100% with aqua bidest. The preferred formulation comprises 10.5% (w/v) Imwitor® 375; 4.5% (w/v) Crodamol™ ethyl oleate (Croda GmbH, Nettetal Kaldenkirchen); 15% (v/v) ethanol; 5% (w/v) 18β-glycyrrhetinic acid and 5% (w/v) (Z)-guggulsterone in aqua bidest. Imwitor® 375; ethyl oleate; 18β-glycyrrhetinic acid and (Z)-guggulsterone were dissolved at room temperature in ethanol, with shaking (500 rpm). Under constant conditions, aqua bidest. was then added slowly and evenly (200 μl/min) to 100% (v/v), and the whole was mixed thoroughly. Finally, the dispersion was homogenised at 200 bar for 5 minutes. The vesicle size was analysed by means of dynamic light scattering using a Zetasizer Nano ZS (Malvern Instruments GmbH, Herrenberg, Germany; for 5 minutes at room temperature) and was in the range 100-150 nm.

### Example 4: Formulation as a Cream

A cream according to the invention preferably comprises flexible soy phosphatidylcholine liposomes containing 18β-glycyrrhetinic acid and (Z)-guggulsterone according to Example 1, which vesicles are incorporated in an amount of 10% (v/w) in a base cream DAC comprising glycerol monostearate 60 4 g; cetyl alcohol 6 g; Miglyol 812 7.5 g; white petroleum jelly (vasilinum album) 25.5 g; Macrogol 20 glyceryl monostearate 7 g; propylene glycol 10 g and aqua bidest. to 100 g. The flexible soy phosphatidylcholine liposomes containing 18β-glycyrrhetinic acid and (Z)-guggulsterone according to Example 1 were thereby incorporated homogeneously into the base cream as the last cream component at 40° C.

### Example 5: Formulation as a Cream

A cream according to the invention preferably comprises oil emulsifier vesicles containing 18β-glycyrrhetinic acid and (Z)-guggulsterone according to Example 2, which vesicles are incorporated in an amount of 10% (v/w) into a cream comprising isooctyl laurate 10 g; cetyl stearyl alcohol 21 g; glycerol (85% v/v) 5 g; propyl hydroxybenzoate 0.05 g; methyl hydroxybenzoate 0.15 g; 0.3 g of limonene; 0.3 g of linanool; ethanol (90% v/v) 1.8 mg and aqua bidest. to 100 g. The oil emulsifier vesicles containing 183-glycyrrhetinic acid and (Z)-guggulsterone were thereby incorporated homogeneously into the cream as the last cream component at 40° C.

### Example 6: Formulation as a Cream

A cream according to the invention preferably comprises emulsifier vesicles containing 18β-glycyrrhetinic acid and (Z)-guggulsterone according to Example 3, which vesicles are incorporated in an amount of 10% (v/w) into a cream comprising PEG-8 L (polyoxyethylene laurate ester) 15 g; ethyl octanoate 5 g; C12-C15 alkyl benzoate 4.5 g; glycerol 3 g; dimethicone 0.5 g; disodium EDTA 0.1 g and aqua bidest. to 100 g. The emulsifier vesicles containing 18β-glycyrrhetinic acid and (Z)-guggulsterone were thereby incorporated homogeneously into the cream as the last cream component at 40° C.

### Example 7: Formulation as a Cream

Oil emulsifier vesicles containing 18β-glycyrrhetinic acid and (Z)-guggulsterone according to Example 2 were incorporated in an amount of 10% (v/w) into a cream comprising 1,2-hexanediol 0.5 g; butylmethoxydibenzoylmethane 4.0 g; Butyrospermum parkii butter (shea butter) 6.0 g; C12-C15 alkyl benzoate 2.0 g; Tego Carbomer 140 0.3 g; cetyl palmitate 1.0 g; cetyl alcohol 1.0 g; cetyl stearyl alcohol 1.0 g; dibutyl adipate 3.0 g; ethylhexyl salicylate 4.5 g; glycerol 10.0 g; glyceryl monostearate 2.5 g; hydrogenated coco-glycerides 1.0 g; methylpropanediol 2.0 g; sodium hyaluronate 0.15 g; perfume; phenoxyethanol 0.6 g; phenylbenzimidazole sulfonic acid 2.0 g; sodium stearyl glutamate 0.2 g; sodium chloride 0.2 g; beeswax 1.0 g; sodium EDTA/sodium hydroxide 2.5 g; aqua bidest. to 100 g. The oil emulsifier vesicles containing 18β-glycyrrhetinic acid and (Z)-guggulsterone were thereby incorporated homogeneously into the cream as the last cream component at 40° C.

### Example 8: Formulation as a Cream

Flexible soy phosphatidylcholine liposomes containing 18β-glycyrrhetinic acid and (Z)-guggulsterone according to Example 1 were incorporated in an amount of 10% (w/v) into a lamellar base cream comprising SLM 2026 (Lipoid AG, Steinhausen, Switzerland) 25 g; caprylic acid triglyceride 18 g; Simmondsia chinensis oil (jojoba oil) 6 g; pentylene glycol 3.75 g; glycerol 3 g; vitamin E acetate 1 g; panthenol 0.5 g; Tego Carbomer 140 0.2 g; sodium hydroxide (20% v/v) 0.15 g; Keltrol CG-SFT 0.1 g; aqua bidest. to 100 g. The flexible soy phosphatidylcholine liposomes containing 18β-glycyrrhetinic acid and (Z)-guggulsterone were thereby incorporated homogeneously into the lamellar base cream as the last cream component at 40° C.

### Example 9: Formulation as a Cream

Flexible soy phosphatidylcholine liposomes containing 18β-glycyrrhetinic acid and (Z)-guggulsterone according to Example 1 were incorporated in an amount of 10% (w/v) into a lamellar base cream comprising SLM 2026 (Lipoid AG, Steinhausen, Switzerland) 30 g; Miglyol 812 15 g; pentylene glycol 10 g; Simmondsia chinensis oil (jojoba oil) 5 g; Butyrospermum parkii butter (shea butter) 2.0 g; squalane 1 g; Phospholipon 90H 1 g; vitamin E acetate 1 g; panthenol 0.5 g; Flowerconcentrole® (Symrise, Holzminden, Germany; mixture of different blossom extracts) 1 g; aqua bidest. to 100 g. The fatty phase and the aqueous phase were first mixed separately at 60° C., with stirring. The fatty phase was homogenised for 3 minutes at 20,000 rpm, and then the aqueous phase was added to the fatty phase, with stirring, and the mixture was homogenised for 5 minutes at 20,000 rpm. After cooling, the flexible soy phosphatidylcholine liposomes containing 18β-glycyrrhetinic acid and (Z)-guggulsterone and the Flowerconcentrole® were incorporated homogeneously into the cream at 40° C. and the pH was adjusted to 5.0-6.0.

### Example 10: Formulation as a Cream

18β-Glycyrrhetinic acid and (Z)-guggulsterone were each incorporated in an amount of 2.5 g into a lamellar base cream comprising SLM 2026 (Lipoid AG, Steinhausen, Switzerland) 30 g; Miglyol 812 15 g; pentylene glycol 10 g; Simmondsia chinensis oil (jojoba oil) 5 g; Butyrospermum parkii butter (shea butter) 2.0 g; squalane 1 g; Phospholipon 90H 1 g; vitamin E acetate 1 g; panthenol 0.5 g; Flowerconcentrole® (Symrise, Holzminden, Germany; mixture of different blossom extracts) 1 g; aqua bidest. to 100 g. The SLM 2026 base cream, the fatty phase and the aqueous phase were first mixed or heated separately at 60° C., with stirring. 18β-Glycyrrhetinic acid and (Z)-guggulsterone were added to the hot fatty phase and the mixture was homogenised intensively, and then the fatty phase was added to the SLM 2026 base cream at 60° C., with stirring, and the mixture was homogenised intensively. The hot aqueous phase was then added at 60° C., with stirring, and the mixture was homogenised for 5 minutes at 20,000 rpm. After cooling, the Flowerconcentrole® was incorporated homogeneously into the cream at 40° C. and the pH was adjusted to 5.0-6.0.

### Example 11: Formulation as a Cream

Flexible soy phosphatidylcholine liposomes containing 18β-glycyrrhetinic acid and (Z)-guggulsterone according to Example 1 were incorporated in an amount of 10% (w/v) into a cream comprising ethylhexyl stearate 10 g; Simmondsia chinensis oil (jojoba oil) 5 g; glyceryl stearate citrate 4 g; pentylene glycol 3 g; decyl oleate 2 g; vitamin E acetate 1 g; Flowerconcentrole® (Symrise, Holzminden, Germany; mixture of different blossom extracts) 1 g; panthenol 0.5 g; cetearyl alcohol 0.5 g; diheptyl succinate 0.5 g; carbomer 0.5 g; aqua bidest. to 100 g. The fatty phase and the aqueous phase were first mixed separately at 60° C., with stirring. The fatty phase was homogenised for 3 minutes at 15,000 rpm, and then the aqueous phase was added to the fatty phase, with stirring, and the mixture was homogenised for 5 minutes at 20,000 rpm. After cooling, the flexible soy phosphatidylcholine liposomes containing 18β-glycyrrhetinic acid and (Z)-guggulsterone and the Flowerconcentrole® were incorporated homogeneously into the cream at 40° C. and the pH was adjusted to 5.0-6.0.

### Example 12: Formulation as a Cream

18β-Glycyrrhetinic acid and (Z)-guggulsterone were each incorporated in an amount of 2.5 g into a lamellar base cream comprising ethylhexyl stearate 8 g; Simmondsia chinensis oil (jojoba oil) 4 g; glyceryl stearate citrate 4 g; decyl oleate 4 g; glycerol 3 g; pentylene glycol 3 g; sucrose stearate 1 g; vitamin E acetate 1 g; Flowerconcentrole® (Symrise, Holzminden, Germany; mixture of different blossom extracts) 1 g; panthenol 0.5 g; cetearyl alcohol 0.5 g; diheptyl succinate 0.4 g; carbomer 0.4 g; acrylate/C10-C30 alkyl acrylate crosspolymer 0.1 g; capryloylglycerol/sebacic acid 0.1 g; trisodium ethylenediamine 0.02 g; aqua bidest. to 100 g. The fatty phase and the aqueous phase were first mixed separately at 60° C., with stirring. The fatty phase was homogenised for 3 minutes at 15,000 rpm and then the aqueous phase was added to the fatty phase, with stirring, and the mixture was homogenised for 5 minutes at 20,000 rpm. After cooling, the Flowerconcentrole® was incorporated homogeneously into the cream at 40° C. and the pH was adjusted to 5.0-6.0. Finally, homogenisation was carried out again for 3 minutes at 15,000 rpm.

### Example 13: Lotion

A lotion according to the invention preferably comprises 20% (w/v) oil emulsifier vesicles containing 18β-glycyrrhetinic acid and (Z)-guggulsterone according to Example 2 as well as glycerol 5 g; unguentum emulsificans aquosum 15 g; propyl hydroxybenzoate 0.3 g; methyl hydroxybenzoate 0.7 g; ethanol (90% v/v) 0.9 g; aqua bidest. to 100 g. The oil emulsifier vesicles containing 18β-glycyrrhetinic acid and (Z)-guggulsterone were thereby incorporated homogeneously into the lotion as the last component at 40° C.

### Example 14: Gel

To 75% (w/v) oil emulsifier vesicles containing 18β-glycyrrhetinic acid and (Z)-guggulsterone according to Example 2 there were added 2% propylene glycol; 0.5% sodium carbomer (PNC 400) and aqua dest. to 100%. The oil emulsifier vesicles containing 18β-glycyrrhetinic acid and (Z)-guggulsterone were thereby incorporated homogeneously into the gel as the last component at 40° C.

### Example 15: Gel

15 g of flexible soy phosphatidylcholine liposomes containing 18β-glycyrrhetinic acid and (Z)-guggulsterone from Example 1 were mixed with 75 grams of a gel base comprising 5 g of mannitol; 0.1 g of sodium edetate; 1 g of propylene glycol; 1 g of Carbopol 934 and 95 g of water. The flexible soy phosphatidylcholine liposomes containing 18β-glycyrrhetinic acid and (Z)-guggulsterone were thereby incorporated homogeneously into the gel as the last component at 40° C.

### Example 16: Gel

18.5 g of flexible soy phosphatidylcholine liposomes containing 18β-glycyrrhetinic acid and (Z)-guggulsterone from Example 1 were processed with 1.75 g of Lipoid PG 14:1, 14:1; 3.75 g of ethanol and 76 g of aqua dest. to form a gel. The flexible soy phosphatidylcholine liposomes containing 18β-glycyrrhetinic acid and (Z)-guggulsterone were thereby incorporated homogeneously into the gel at 40° C.

### Example 17: Gel

18β-Glycyrrhetinic acid and (Z)-guggulsterone were each incorporated in an amount of 2.5 g into a phospholipid gel consisting of 13.5 g of Phospholipon 90G; 1.5 g of Lipoid PG 14:1, 14:1; 3.75 g of ethanol and 76.25 g of aqua dest. To that end, Phospholipon 90G was dissolved in ethanol with heating and stirring (60° C./100 rpm). 18β-Glycyrrhetinic acid and (Z)-guggulsterone were melted with Lipoid PG 14:1, 14:1 at 60° C. and mixed homogeneously. The dissolved Phospholipon 90G was then added and made into a paste at 60° C. with the ingredients already present. During cooling to 45° C., aqua dest. was added stepwise and stirring was carried out slowly to form a gel. The gel was then left to swell at 45° C. before being stirred slowly for a further 5 minutes at 45° C. to give a homogeneous gel.

### Example 18: Vesicles Based on a Lipidic Emulsifier from the Food Additives Group, a Monoester of a Long-Chained Fatty Acid and a Plant Oil and Comprising an Active Ingredient Combination of 18β-Glycyrrhetinic Acid and (Z)-Guggulsterone

The preferred formulation comprises vesicles which comprise 8.5% (w/w) soybean oil; 3.5% (w/w) Imwitor® 375; 10% (w/w) ethyl oleate; 10% (w/w) ethanol; 1.5% (w/w) diglyceryl monooleate; 0.75% (w/w) 18β-glycyrrhetinic acid and 0.75% (w/w) (Z)-guggulsterone in aqua bidest. (ad 100%).

Also preferred are vesicles which comprise either 18β-glycyrrhetinic acid or (Z)-guggulsterone in an amount of 1.5% (w/w):

(a) 1.5% (w/w) 18β-glycyrrhetinic acid with 13.5% (w/w) sunflower seed oil; 3.5% (w/w) Imwitor® 375; 5% (w/w) ethyl oleate; 10% (w/w) ethanol; 1.5% (w/w) diglyceryl monooleate in aqua bidest. (ad 100%).

(b) 1.5% (w/w) (Z)-guggulsterone with 8.5% (w/w) sunflower seed oil; 3.5% (w/w) Imwitor® 375; 10% (w/w) ethyl oleate; 10% (w/w) ethanol; 1.5% (w/w) diglyceryl monooleate in aqua bidest. (ad 100%).

Both vesicle types, (a) and (b), are preferably added to the final formulation in the ratio 1:1. The plant oil (soybean or sunflower oil), Imwitor® 375, ethyl oleate, diglyceryl monooleate, 18β-glycyrrhetinic acid and (Z)-guggulsterone were dissolved at room temperature in ethanol, with shaking (500 rpm). Under constant conditions, aqua bidest. was then added slowly and evenly (200 μl/min) to 100% (v/v), and the whole was mixed thoroughly. Finally, the dispersion was vortexed for 5 minutes, treated with ultrasound for 5 minutes (on/off interval: 10 s) and extruded through a PC filter membrane (200 nm pore size). The vesicle size was analysed by means of dynamic light scattering using a Zetasizer Nano ZS (Malvern Instruments GmbH, Herrenberg, Germany; for 5 minutes at room temperature) and was in the range 80-140 nm.

### Example 19: Vesicles Based on a Lipidic Emulsifier from the Food Additives Group and a Monoester of a Long-Chained Fatty Acid and Comprising an Active Ingredient Combination of 18β-Glycyrrhetinic Acid and (Z)-Guggulsterone

Preference is given to vesicles which comprise either 18β-glycyrrhetinic acid or (Z)-guggulsterone in an amount of 1.5% (w/w):

(a) 1.5% (w/w) 18β-glycyrrhetinic acid with 10.5% (w/w) Imwitor® 375; 4.5% (w/w) ethyl oleate; 16% (w/w) ethanol in aqua bidest. (ad 100%).

(b) 1.5% (w/w) (Z)-guggulsterone with 14% (w/w) Imwitor® 375; 6% (w/w) ethyl oleate; 16% (w/w) ethanol in aqua bidest. (ad 100%).

Both vesicle types, (a) and (b), are preferably added to the final formulation in the ratio 1:1. Imwitor® 375, ethyl oleate, 18β-glycyrrhetinic acid and (Z)-guggulsterone were dissolved at room temperature in ethanol, with shaking (500 rpm). Under constant conditions, aqua bidest. was then added slowly and evenly (200 μl/min) to 100% (v/v), and the whole was mixed thoroughly. Finally, the dispersion was vortexed for 5 minutes, treated with ultrasound for 5 minutes (on/off interval: 10 s) and extruded through a PC filter membrane (200 nm pore size). The vesicle size was analysed by means of dynamic light scattering using a Zetasizer Nano ZS (Malvern Instruments GmbH, Herrenberg, Germany; for 5 minutes at room temperature) and was in the range 80-160 nm.

### Example 20: Vesicles Based on Synthetic Cetyl Palmitate, a Plant Oil, a Plant Polysaccharide Copolymer and a Stearic-Acid-Based Emulsifier and Comprising an Active Ingredient Combination of 18β-Glycyrrhetinic Acid and (Z)-Guggulsterone

The preferred formulation comprises vesicles of 20% (w/w) cetyl palmitate; 10% (w/w) jojoba oil; 5% (w/w) 18β-glycyrrhetinic acid; 5% (w/w) (Z)-guggulsterone; 1.5% (w/w) TEGO® Care PS and 0.5% (w/w) Inutec® SP1 in aqua bidest. (ad 100%).

Also preferred are vesicles which comprise either 18β-glycyrrhetinic acid or (Z)-guggulsterone in an amount of 5% (w/w):

(a) 5% (w/w) 18β-glycyrrhetinic acid with 20% (w/w) cetyl palmitate; 10% (w/w) jojoba oil; 1.5% (w/w) TEGO® Care PS and 0.5% (w/w) Inutec® SP1 in aqua bidest. (ad 100%).

(b) 5% (w/w) (Z)-guggulsterone with 20% (w/w) cetyl palmitate; 10% (w/w) jojoba oil; 1.5% (w/w) TEGO® Care PS and 0.5% (w/w) Inutec® SP1 in aqua bidest. (ad 100%).

Both vesicle types, (a) and (b), are preferably added to the final formulation in the ratio 1:1. 18β-Glycyrrhetinic acid or (Z)-guggulsterone were resuspended in jojoba oil, with stirring. The cetyl palmitate was melted at 60° C.; TEGO® Care PS and the jojoba oil containing glycyrrhetinic acid and/or guggulsterone were added, with intensive stirring. Inutec® SP1 was dissolved at 60° C. in purified water. At 60° C., the lipid mixture was added gradually to the Inutec® SP1 solution. The dispersion was homogenised at 60° C. by means of a high-pressure homogeniser and finally cooled to room temperature.

### Example 21: Formulation as a Cream Face Mask

18β-Glycyrrhetinic acid and (Z)-guggulsterone were each incorporated in an amount of 2.5 g into a lamellar base cream comprising SLM 2026 (Lipoid AG, Steinhausen, Switzerland) 86.5 g; Simmondsia chinensis oil (jojoba oil) 6 g; vitamin E acetate 1 g; panthenol 0.5 g; Flowerconcentrole® mix (blossom extracts; Symrise, Holzminden, Germany) 1 g. The ingredients were thereby incorporated homogeneously into the lamellar base cream stepwise at 40° C.

### Example 22: Formulation as a Cream Face Mask

Oil emulsifier vesicles containing 18β-glycyrrhetinic acid and (Z)-guggulsterone according to Example 18 were incorporated in an amount of 10% (v/w) into a lamellar base cream comprising SLM 2026 (Lipoid AG, Steinhausen, Switzerland) 81.5 g; Simmondsia chinensis oil (jojoba oil) 6 g; vitamin E acetate 1 g; panthenol 0.5 g; Flowerconcentrole® mix (blossom extracts; Symrise, Holzminden, Germany) 1 g. The oil emulsifier vesicles containing 18β-glycyrrhetinic acid and (Z)-guggulsterone were thereby incorporated homogeneously into the base cream at 40° C. as the last cream component.

### Example 23: Formulation as a Cream Face Mask

A cream face mask according to the invention preferably comprises 2.5 g of each of 18β-glycyrrhetinic acid and (Z)-guggulsterone, which are incorporated into a base cream DAC comprising glycerol monostearate 60 4 g; cetyl alcohol 6 g; Miglyol 812 7.5 g; white petroleum jelly (vasilinum album) 25.5 g; Macrogol 20 glyceryl monostearate 7 g; propylene glycol 10 g and aqua bidest. to 100 g. The cream face mask further comprises vitamin E acetate 1 g; Simmondsia chinensis oil (jojoba oil) 5 g and Flowerconcentrole® mix (blossom extracts; Symrise, Holzminden, Germany) 1 g. The ingredients were thereby incorporated homogeneously into the base cream stepwise at 40° C.

### Example 24: Formulation as a Cream Face Mask

Emulsifier vesicles containing 18β-glycyrrhetinic acid and (Z)-guggulsterone according to Example 19 were incorporated in an amount of 10% (v/w) into a base cream DAC comprising glycerol monostearate 60 4 g; cetyl alcohol 6 g; Miglyol 812 7.5 g; white petroleum jelly (vasilinum album) 25.5 g; Macrogol 20 glyceryl monostearate 7 g; propylene glycol 10 g and aqua bidest. to 100 g. The cream face mask further comprises vitamin E acetate 1 g; ethanol 5 g and Flowerconcentrole® mix (blossom extracts; Symrise, Holzminden, Germany) 1 g. The emulsifier vesicles containing 18β-glycyrrhetinic acid and (Z)-guggulsterone were thereby incorporated homogeneously into the base cream as the last cream component at 40° C.

### Example 25: Formulation as a Cream Face Mask

Vesicles containing 18β-glycyrrhetinic acid and (Z)-guggulsterone according to Example 20 were incorporated in an amount of 10% (v/w) into a base cream DAC comprising glycerol monostearate 60 4 g; cetyl alcohol 6 g; Miglyol 812 7.5 g; white petroleum jelly (vasilinum album) 25.5 g; Macrogol 20 glyceryl monostearate 7 g; propylene glycol 10 g and aqua bidest. to 100 g. The cream face mask further comprises vitamin E acetate 1 g; panthenol 0.5 g and Flowerconcentrole® mix (blossom extracts; Symrise, Holzminden, Germany) 1 g. The vesicles containing 18β-glycyrrhetinic acid and (Z)-guggulsterone were thereby incorporated homogeneously into the cream as the last cream component at 40° C.

### Example 26: Formulation as a Cream Face Mask

Oil emulsifier vesicles containing 18β-glycyrrhetinic acid and (Z)-guggulsterone according to Example 18 were incorporated in an amount of 10% (v/w) into a cream comprising PEG-8 L (polyoxyethylene laurate ester) 15 g; ethyl octanoate 5 g; C12-C15 alkyl benzoate 4.5 g; glycerol (85%) 3 g; propylene glycol 3 g; vitamin E acetate 1 g; dimethicone 350 0.5 g, Flowerconcentrole® mix (blossom extracts; Symrise, Holzminden, Germany) 1 g and aqua bidest. to 100 g. The oil emulsifier vesicles containing 18β-glycyrrhetinic acid and (Z)-guggulsterone were thereby incorporated homogeneously into the formulation as the last component at 40° C.

### Example 27: Formulation as a Gel Face Mask

A gel face mask according to the invention preferably comprises 2.5 g of each of 18β-glycyrrhetinic acid and (Z)-guggulsterone; ethanol 5 g; mannitol 5 g; sodium edetate 1 g; propylene glycol 1 g; vitamin E acetate 1 g; Carbopol 981 0.5 g; Flowerconcentrole® mix (blossom extracts; Symrise, Holzminden, Germany) 1 g and aqua bidest. to 100 g. The ingredients were thereby incorporated homogeneously into the formulation stepwise at 40° C.

### Example 28: Formulation as a Gel Face Mask

A gel face mask according to the invention preferably comprises 2.5 g of each of 180-glycyrrhetinic acid and (Z)-guggulsterone; 2-propanol 10 g; vitamin E acetate 1 g; Carbopol 981 0.5 g; Flowerconcentrole® mix (blossom extracts; Symrise, Holzminden, Germany) 1 g and aqua bidest. to 100 g. The ingredients were thereby incorporated homogeneously into the formulation stepwise at 40° C.

### Example 29: Formulation as an Ointment Face Mask

An ointment face mask according to the invention preferably comprises 2.5 g of each of 180-glycyrrhetinic acid and (Z)-guggulsterone; ethanol 5 g; vitamin E acetate 1 g; unguentum emulsificans aquosum N SR (comprising non-ionic, emulsifying alcohols 21 g; 2-ethylhexyl laurate 10 g; glycerol (85%) 5 g; potassium sorbate 0.14 g; citrate (anhydrous) 0.07 g; aqua ad 100 g); Flowerconcentrole® mix (blossom extracts; Symrise, Holzminden, Germany) 1 g and aqua bidest. to 100 g. The ingredients were thereby incorporated homogeneously into the formulation stepwise at 40° C.

### Example 30: Formulation as an Ointment Face Mask

An ointment face mask according to the invention preferably comprises 2.5 g of each of 18β-glycyrrhetinic acid and (Z)-guggulsterone; Simmondsia chinensis oil (jojoba oil) 5 g; vitamin E acetate 1 g; Linimentum aquosum N (comprising emulsifying cetyl stearyl alcohol (type A) 11.5 g; 2-ethylhexyl laurate 5 g; glycerol (85%) 2.5 g; potassium sorbate 0.07 g; citrate (anhydrous) 0.035 g; aqua to 100 g); Flowerconcentrole® mix (blossom extracts; Symrise, Holzminden, Germany) 1 g and aqua bidest. to 100 g. The ingredients were thereby incorporated homogeneously into the formulation stepwise at 40° C.

### Example 31: Formulation as a Lotion Face Mask

A lotion face mask according to the invention preferably comprises 2.5 g of each of 18β-glycyrrhetinic acid and (Z)-guggulsterone; ethanol 2 g; vitamin E acetate 1 g; panthenol 0.5 g; glycerol (85%) 5 g; Pionier 1033 7 g; viscous paraffin 20 g; urea 2 g; white petroleum jelly 20 g; magnesium sulfate heptahydrate 0.5 g; methyl hydroxybenzoate 0.3 g; propyl hydroxybenzoate 0.7 g; Flowerconcentrole® mix (blossom extracts; Symrise, Holzminden, Germany) 1 g and aqua bidest. to 100 g. The ingredients were thereby incorporated homogeneously into the formulation stepwise at 40° C.

The face masks of Examples 27 to 31 were further prepared using the vesicles of Examples 16, 17 and 18.

The creams, lotions and gels of Examples 4 to 17 can further be prepared using the vesicles of Examples 18, 19 and 20.

### Example 32: Further Examples of Compositions of Face Masks

Ingredients composition A:
Purified water
Bentonite
Glycerol
Acacia senegal gum
Xanthan gum
Benzyl alcohol
Ethanol
Sodium dehydroacetate
Simmondsia chinensis oil (jojoba oil)
Sodium citrate
Dehydroacetic acid
Citric acid
Blossom extract (fragrance)
Carbomer
1,2-Hexanediol
Caprylyl glycol
Sodium benzoate
Lactic acid
Potassium sorbate
Algin
Panthenol
Tocopheryl acetate

Ingredients composition B:
Purified water
Simmondsia chinensis oil (jojoba oil)
Butyrospermum parkii (shea) butter extract
Glycerol
Glyceryl stearate
Cetearyl alcohol
Cera alba (beeswax)
Caprylic/capric triglyceride
Benzyl alcohol
Sodium salicylate
Blossom extract (fragrance)
Levulinic acid
Xanthan gum
Sodium levulinate
Sodium hydroxide
Dehydroacetic acid
Sodium benzoate
Potassium sorbate
Citric acid
Panthenol
Tocopheryl acetate

Ingredients composition C:
Purified water
Caprylic/capric triglyceride
Hectorite
Cetearyl alcohol
Pentylene glycol
Isopropyl isostearate
Glycerol
Glyceryl stearate
Ceteareth-20
Panthenol
Tocopherol
Tocopheryl acetate
Benzyl alcohol
Allantoin
Ceteareth-12
Cetyl palmitate
Blossom extract (fragrance)
Citric acid
Benzoic acid
Sodium hyaluronate (0.01-0.5%)
Sorbic acid
Benzyl alcohol

To these compositions A, B or C there are added either 18β-glycyrrhetinic acid and (Z)-guggulsterone or the vesicles according to the invention.

1. Vesicles comprising:
a) at least one glycyrrhetinic acid or a salt or an ester thereof;
b) at least one guggulsterone; and
c) either: c1) phosphatidylcholine, or
c2) at least one emulsifier from the food additives group and at least one monoester of a long-chained fatty acid.

2. Vesicles according to claim 1, comprising:
a) at least one glycyrrhetinic acid or a salt or an ester thereof;
b) at least one guggulsterone; and
c) phosphatidylcholine.

3. Vesicles according to claim 1, comprising:
a) at least one glycyrrhetinic acid or a salt or an ester thereof;
b) at least one guggulsterone; and
c) at least one emulsifier from the food additives group and at least one monoester of a long-chained fatty acid.

4. Vesicles according to claim 1, wherein the phosphatidylcholine originates from soy lecithin or sunflower lecithin.

5. Vesicles according to claim 1, wherein the phosphatidylcholine is used in the form of soy lecithin or sunflower lecithin.

6. Vesicles according to claim 3, which further comprise a plant oil.

7. Vesicles according to claim 1 wherein the at least one glycyrrhetinic acid or the salt or the ester thereof is chosen from 18α-glycyrrhetinic acid and/or 18β-glycyrrhetinic acid or one or more salt(s) or ester(s) thereof, and in that the at least one guggulsterone is selected from (Z)-guggulsterone and/or (E)-guggulsterone.

8. Vesicles according to claim 1 wherein the at least one glycyrrhetinic acid or the salt or the ester thereof is used in the form of Glycyrrhiza glabra or in the form of extract from Glycyrrhiza glabra and/or in the form of a pure chemical substance and/or in the form of salts or esters thereof, and/or in that the at least one guggulsterone is used in the form of guggul lipid or in the form of extract from guggul lipid and/or in the form of a pure chemical substance.

9. Vesicles according to claim 1 wherein the at least one emulsifier from the food additives group is selected from the food additives E471, E472a, E472b, E472c, E472d, E472e, E472f, E473, E474, E475, E476 and E477.

10. Vesicles according to claim 1, comprising:
0.1-0.5 part by weight of a glycyrrhetinic acid or of a salt or an ester thereof;
0.1-0.5 part by weight of a guggulsterone; and
0.5-3 parts by weight of at least one surfactant;
per 10 parts by weight of phosphatidylcholine.

11. Vesicles according to claim 1, comprising:
15-20% (v/v) seed oil of an edible plant;
2.5-10% (w/v) emulsifier(s) from the food additives group;
2.5-5% (w/v) of at least one monoester of a long-chained fatty acid;
10-20% (v/v) ethanol;
0.1-10% (w/v) 18β-glycyrrhetinic acid; and
0.1-10% (w/v) (Z)-guggulsterone;
made up to 100% with water.

12. Vesicles according to claim 1, comprising:
7-17.5% (w/v) emulsifier(s) from the food additives group;
3-7.5% (w/v) of at least one monoester of a long-chained fatty acid;
10-20% (v/v) ethanol;
0.1-10% (w/v) 18β-glycyrrhetinic acid; and
0.1-10% (w/v) (Z)-guggulsterone;
made up to 100% with water.

13. Cream, lotion or gel comprising vesicles according to claim 1.

14. Cream, lotion or gel according to claim 13, characterised in that the at least one glycyrrhetinic acid or the salt or the ester thereof and the at least one guggulsterone are each present in an amount of 0.01-10% (w/w), preferably 0.1-5% (w/w), based on the total weight of the cream, lotion or gel.

15. Vesicles comprising:
0-5% (w/w) glycyrrhetinic acid or a salt or an ester thereof
0-5% (w/w) guggulsterone
5-25% (w/w) abs. ethanol
1-30% (w/w) ethyl oleate
0-30% (w/w) plant oil (for example sunflower oil, soybean oil, rape oil)
0.5-5% (w/w) diglyceryl monooleate
0-5% (w/w) Imwitor 375 (glyceryl citrate/lactate/linoleate/oleate)
ad 100% purified water;
wherein the vesicles comprise at least glycyrrhetinic acid or a salt or an ester thereof or guggulsterone.

16. Vesicles comprising:
0-5% (w/w) glycyrrhetinic acid or a salt or an ester thereof
0-5% (w/w) guggulsterone
5-25% (w/w) abs. ethanol
1-10% (w/w) ethyl oleate
5-25% (w/w) Imwitor 375 (glyceryl citrate/lactate/linoleate/oleate)
ad 100% purified water;
wherein the vesicles comprise at least glycyrrhetinic acid or a salt or an ester thereof or guggulsterone.

17. Vesicles comprising:
0-5% (w/w) glycyrrhetinic acid or a salt or an ester thereof
0-5% (w/w) guggulsterone
10-15% (w/w) jojoba oil
15-25% (w/w) cetyl palmitate
0.2-1.0% (w/w) Inutec® SP1
1-3% (w/w) TEGO® Care PS
ad 100% purified water;
wherein the vesicles comprise at least glycyrrhetinic acid or a salt or an ester thereof or guggulsterone.

18. Formulation of the vesicles according to claim 1 as a cream, a lotion or a gel for use in the cosmetic and/or dermatological treatment and/or prophylaxis of cellulite and/or for the treatment and/or prophylaxis of the appearance of ageing skin.

19. Face mask comprising at least one glycyrrhetinic acid or a salt or an ester thereof and at least one guggulsterone.

20. Face mask comprising a vesicle according to claim 1.

21. Face mask comprising at least one glycyrrhetinic acid or a salt or an ester thereof and at least one guggulsterone each in an amount of 0.01-5% (w/w), preferably 0.1-2.5% (w/w), based on the total weight of the face mask.

22-23. (canceled)

24. Plaster or patch comprising at least one glycyrrhetinic acid or a salt or an ester thereof and at least one guggulsterone.

25. Plaster or patch comprising a vesicle according to claim 1.

26. Composition comprising:
A) vesicles comprising:
a) at least one glycyrrhetinic acid or a salt or an ester thereof; and
c) either: c1) phosphatidylcholine, or
c2) at least one emulsifier from the food additives group and at least one monoester of a long-chained fatty acid; and
B) vesicles comprising:
b) at least one guggulsterone; and
d) either: d1) phosphatidylcholine, or
d2) at least one emulsifier from the food additives group and at least one monoester of a long-chained fatty acid.

27. Cream, lotion, gel, plaster, patch or face mask comprising a composition according to claim 26.

28. Formulation of the composition according to claim 26 as a cream, a lotion or a gel for use in the cosmetic and/or dermatological treatment and/or prophylaxis of cellulite and/or for the treatment and/or prophylaxis of the appearance of ageing skin.

29-30. (canceled)

31. A method for treatment of prophylaxis of ageing or stressed skin comprising administering to a subject vesicles according to claim 1.

32. A method of treatment of a changes in subcutaneous fatty or connective tissue comprising administering to a subject vesicles according to claim 1 or of a combination of at least one glycyrrhetinic acid or a salt or an ester thereof and at least one guggulsterone.

33. The method of claim 32 wherein the changes in subcutaneous fatty or connective tissue are lipoedema, lipoma, lipomatosis of the abdominal wall, dermatopanniculosis deformans, pseudogynecomastia, buffalo hump in HIV patients, and non-specific subcutaneous fat deposits, or in the regression or reduction of fat pads which are not caused by disease, such as, for example, fat pads in the face and neck region (for example lachrymal sacs, nasolabial folds, flabby cheeks, double chin, etc.), or in after-treatment following liposuction.

34. A method for treatment of prophylaxis of ageing or stressed skin comprising administering to a subject a composition of claim 26.

35. A method of treatment of a changes in subcutaneous fatty or connective tissue comprising administering to a subject a composition of claim 26.

36. The method of claim 35 wherein the changes in subcutaneous fatty or connective tissue are lipoedema, lipoma, lipomatosis of the abdominal wall, dermatopanniculosis deformans, pseudogynecomastia, buffalo hump in HIV patients, and non-specific subcutaneous fat deposits, or in the regression or reduction of fat pads which are not caused by disease, such as, for example, fat pads in the face and neck region (for example lachrymal sacs, nasolabial folds, flabby cheeks, double chin, etc.), or in after-treatment following liposuction.
